(19) United States
(12) Patent Application Publication
(10) Pub. No.: US 2022/0283176 A1
(43) Pub. Date: Sep. 8, 2022

(54) METHOD FOR THE ABSOLUTE QUANTIFICATION OF MHC MOLECULES

(71) Applicant: Immatics Biotechnologies GmbH, Tuebingen (DE)

(72) Inventors: Christoph SCHRAEDER, Tuebingen (DE); Heiko SCHUSTER, Tuebingen (DE); Lena Katharina FREUDENMANN, Tuebingen (DE); Lida ROSTOCK, Tuebingen (DE); Linus BACKERT, Tuebingen (DE); Michael ROEMER, Tuebingen (DE); Daniel Johannes KOWALEWSKI, Tuebingen (DE)

(21) Appl. No.: 17/686,224

(22) Filed: Mar. 3, 2022

Related U.S. Application Data
(60) Provisional application No. 63/156,136, filed on Mar. 3, 2021.

(30) Foreign Application Priority Data
Mar. 3, 2021 (DE) 10 2021 105 142.8

(51) Int. Cl. G01N 33/68; G01N 1/34

(52) U.S. Cl. CPC G01N 33/6842; G01N 33/6875; G01N 33/6848; G01N 1/34; G01N 2333/70539

(57) ABSTRACT

The present invention relates to a method for the absolute quantification of one or more MHC molecules in a test sample comprising at least one cell, the method comprising at least the steps of: homogenizing the sample, adding an internal standard to the sample, digesting the homogenized sample with a protease, before or after addition of the internal standard, purifying the sample obtained by the digestion, subjecting the digested sample to a step of chromatography and/or spectrometry analysis, and quantifying the one or more MHC molecules in the test sample Also, the invention relates to method of determining the cell count in a sample. (FIG. 1).

### CROSS-REFERENCE TO RELATED APPLICATIONS

This application claims priority to U.S. Provisional Application No. 63/156,136, filed Mar. 3, 2021, and German Patent Application No. 10 2021 105 142.8, filed Mar. 3, 2021. Each of these applications is incorporated by reference in its entirety.

### REFERENCE TO SEQUENCE LISTING SUBMITTED AS A COMPLIANT ASCII TEXT FILE (.txt)

Pursuant to the EFS-Web legal framework and 37 C.F.R. § 1.821-825 (see M.P.E.P. § 2442.03(a)), a Sequence Listing in the form of an ASCII-compliant text file (entitled “2912919-107001_Sequence_Listing_ST25.txt” created on Mar. 2, 2022, and 28,118 bytes in size) is submitted concurrently with the instant application, and the entire contents of the Sequence Listing are incorporated herein by reference.

### FIELD OF THE INVENTION

The present application relates to a method for the absolute quantification of MHC molecules.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present specification are intended.

### BACKGROUND

The major histocompatibility complex (MHC) is a gene cluster on chromosome 6 which is common to most vertebrates encoding for different genes, which play a fundamental role in histocompatibility and the adaptive immune system. In humans this cluster is often also commonly referred to as human leukocyte antigen (HLA). MHC class I molecules are expressed on all cells of a mammal with the exception of erythrocytes. Their main function is to present short peptides derived from intracellular or endocytosed proteins to cytotoxic T lymphocytes (CTLs) (Boniface and Davis, 1995; Goldberg and Rizzo, 2015b; Gruen and Weissman, 1997; Rock and Shen, 2005). CTLs express CD8 co-receptors, in addition to T cell receptors (TCRs). When a CTL's CD8 receptor docks to an MHC class I molecule on a target cell, if the CTL's TCR fits the epitope represented by the complex of MHC class I molecule and presented peptide, the CTL triggers the target cell lysis by either releasing a cargo of cytolytic enzymes or rendering the cell to undergo programmed cell death by apoptosis (Boniface and Davis, 1995; Delves and Roitt, 2000; Lustgarten et al., 1991). Thus, MHC class I helps mediate cellular immunity, a primary means to address intracellular pathogens, such as viruses and some bacteria, including bacterial L forms or bacterial genera Shigella and Rickettsia (Goldberg and Rizzo, 2015b; Madden et al., 1993; Ray et al., 2009). Furthermore this process is also of utmost importance for the immunological response and defense against neoplastic diseases such as cancer (Coley, 1991; Coulie et al., 2014; Urban and Schreiber, 1992).

Heterodimeric MHC class I molecules are composed of a polymorphic heavy α-subunit encoded within the MHC gene cluster and a small invariant beta-2-microglobulin (β2m) subunit whose gene is located outside of the MHC locus on chromosome 15. The polymorphic a chain encompasses an N-terminal extracellular region composed by three domains, α1, α2, and α3, a transmembrane helix accomplishing cell surface attachment of the MHC molecule, and a short cytoplasmic tail. Two domains, α1 and α2, form a peptide-binding groove between two long α-helices, whereas the floor of the groove is formed by eight β-strands. The Immunoglobulin-like domain α3 is involved in the interaction with the CD8 co-receptor. The invariant (32m provides stability of the complex and participates in recognition of the peptide-MHC class I complex by CD8 co-receptors. (32m is non-covalently bound to the α-subunit. It is held by several pockets on the floor of the peptide-binding groove. Amino acid (AA) side chains that vary widely between different human HLA alleles fill up the central and widest portion of the binding groove, while conserved side chains are clustered at the narrower ends of the groove. The polymorphic amino acid residues authoritatively define the biochemical properties of peptides which can be bound by the respective HLA molecule (Boniface and Davis, 1995; Falk et al., 1991; Goldberg and Rizzo, 2015a; Rammensee et al., 1995).

In humans, the MHC class I gene cluster is characterized by polymorphism and polygenicity. Each chromosome encodes one HLA-A, -B, and -C allele together constituting the HLA class I haplotype. Consequently, up to six different classical HLA class I molecules can be expressed on the surface of an individual's cells; an exemplary combination of HLA-A, -B, and -C allotypes is given in the table below. In December 2020, the IPD-IMGT/HLA Database (release 3.42.0, 2020-10-15) comprised a total of 6,291 HLA-A alleles (3,896 proteins), 7,562 HLA-B alleles (4,803 proteins), and 6,223 HLA-C alleles (3,618 proteins) (Robinson et al., 2015).

| HLA-A | HLA-B | HLA-C |
|---|---|---|
| A*02:01 | B*40:02 | C*03:04 |
| A*24:02 | B*52:01 | C*12:02 |

In multifactorial disease development, genetic predisposition represents a common element enclosing, inter alia, the composition of an individual's HLA alleles. Autoimmune disorders such as ankylosing spondylitis (HLA-B*27), celiac disease (HLA-DQA1*05:01-DQB1*02:01 or HLA-DQA1*03:01-DQB1*03:02), narcolepsy (HLA-DQB1*06:02), or type 1 diabetes (HLA-DRB1*04:01-DQB1*03:02) have a long history of HLA association (Caillat-Zucman, 2009). Moreover, it has become evident that specific HLA allotypes have an influence on the risk of contagion as well as the course of infections e.g. with the human immunodeficiency virus or malaria parasites (Hill et al., 1991; The International HIV Controllers Study et al., 2010; Trachtenberg et al., 2003). Besides that, the individual HLA genotype shapes the response to cancer immunotherapy: while maximal heterozygosity of HLA-A, -B, and -C alleles appears to favor the response to checkpoint blockade, HLA-B*15:01 has been suggested to impair neo-antigen-directed CTL responses (Chowell et al., 2018).

MHC molecules are tissue antigens that allow the immune system to bind to, recognize, and tolerate itself (autorecognition). MHC molecules also function as chaperones for intracellular peptides that are complexed with MHC heterodimers and presented to T cells as potential foreign antigens (Felix and Allen, 2007; Stern and Wiley, 1994).

MHC molecules interact with TCRs and different co-receptors to optimize binding conditions for the TCR-antigen interaction, in terms of antigen binding affinity and specificity, and signal transduction effectiveness (Boniface and Davis, 1995; Gao et al., 2000; Lustgarten et al., 1991). Essentially, the MHC-peptide complex is a complex of auto-antigen/allo-antigen. Upon binding, T cells should in principle tolerate the auto-antigen, but activate when exposed to the allo-antigen. Disease states (especially autoimmunity) occur when this principle is disrupted (Basu et al., 2001; Felix and Allen, 2007; Whitelegg et al., 2005).

On MHC class I, a cell normally presents cytosolic peptides, mostly self-peptides derived from protein turnover and defective ribosomal products (Goldberg and Rizzo, 2015b; Schwanhausser et al., 2011, 2013; Yewdell, 2003; Yewdell et al., 1996). These peptides typically have an extended conformation and oftentimes a length of 8 to 12 amino acids residues, but accommodation of slightly longer versions is feasible as well (Guo et al., 1992; Madden et al., 1993; Rammensee, 1995). During infection with intracellular pathogens including viruses and microorganisms as well as in the course of cancerous transformation, proteins of foreign origin or associated with malignant transformation are also degraded in the proteasome, loaded onto MHC class I molecules, and further displayed on the cell surface (Goldberg and Rizzo, 2015b; Madden et al., 1993; Urban and Schreiber, 1992). Moreover, a phenomenon designated as cross-presentation accomplishes loading of extracellular antigens on MHC class I enabling activation of naïve CTLs by dendritic cells (DCs) (Rock and Shen, 2005). T cells can detect a peptide displayed at 0.1%-1% of the MHC molecules and still evoke an immune reaction (Davenport et al., 2018; Sharma and Kranz, 2016; Siller-Farfan and Dushek, 2018; van der Merwe and Dushek, 2011).

Depending on their origin, the peptides displayed by MHC class I are called “tumor-associated peptides” (TUMAPs), “virus-derived peptides” or, more general, “pathogen-derived peptides” (Coulie et al., 2014; Freudenmann et al., 2018; Kirner et al., 2014; Urban and Schreiber, 1992).

The interplay between MHC class I, peptides presented thereby, and T cell receptors has been used as a leverage for therapeutic interventions, including (i) vaccination, (ii) TCR therapy, and (iii) adoptive T-cell therapy (Dahan and Reiter, 2012; He et al., 2019; Hilf et al., 2019; Kuhn et al., 2019; Rosenberg et al., 2011; Velcheti and Schalper, 2016).

Vaccination with TUMAPs has been used to prime and activate the immune system against cancer. The underlying activation cascade comprises vaccination, priming, proliferation and elimination. In the vaccination step, TUMAPs are administered intradermally together with adjuvants/immunomodulators to create an inflammatory milieu and recruit and mature immune cells (dendritic cells). In the priming step, TUMAPs are again administered and bind to dermal DCs, where they are loaded onto MHC class I molecules. The DCs then migrate into the lymph nodes, where they activate (“prime”) naïve T cells specifically recognizing the TUMAPs used in the vaccine via their TCR. Once T cells are primed, their number increases rapidly (clonal proliferation). They leave the lymph nodes and begin searching for tumor cells displaying exactly the same TUMAP on their MHCs by which they were activated in the process of priming. Once a respective target cell is found, the T cell mounts a cytolytic/apoptotic attack against the tumor cells (Hilf et al., 2019; Kirner et al., 2014; Molenkamp et al., 2005).

An alternative category of therapeutic approaches employs engineered, soluble TCRs recognizing a specific pathogen-derived or tumor-associated peptide when presented on MHC (Dahan and Reiter, 2012; He et al., 2019). These TCRs may carry an immunomodulatory moiety that is capable of engaging T cells, like an fragment that has affinity to CD3, a molecule that is abundant on T cells. By this mechanism, T cells are redirected to the site of disease and mount a cytolytic/apoptotic attack against the target cells (Chang et al., 2016; Dao et al., 2015; He et al., 2019). A major advantage of soluble TCRs over antibody-based (immuno) therapies is the expansion of the potential target repertoire to intracellular proteins instead of being limited to cell surface antigens accessible to classical antibody formats (Dahan and Reiter, 2012; He et al., 2019).

In adoptive T-cell therapy, a patient's own T cells are isolated, optionally enriched for clones with desired antigen specificity, expanded in vitro, and re-infused into the patient. Isolated autologous T cells can further be modified to express a TCR that has been engineered to recognize a specific pathogen-derived or tumor-associated peptide. In such way, these T cells are taught to bind to cells at the site of disease and exert a cytolytic/apoptotic attack against these target cells. Moreover, it is possible to incorporate co-stimulatory molecules such as CD40 ligand into these T cells equipped with chimeric antigen receptors (CAR) to further enhance the triggered anti-tumor immune response (Kuhn et al., 2019; Rosenberg et al., 2011).

In all these approaches, MHC class I is a critical element. In order to better assess the quantitative and qualitative relevance of MHC class I for a given therapeutic approach, it would be desirable to be able to absolutely quantify a given MHC class I subtype in the present sample.

This would be extremely helpful to be able to, for example,
a) predict a therapeutic window for one of the therapeutic modalities discussed above, and/or
b) determine whether or not a given MHC subtype is expressed in a sample of interest, to be able to assess whether or not a given therapeutic modality is applicable, and/or
c) assess whether or not a given disease state or an applied therapeutic modality is associated with quantitative changes of MHC levels.

Caron et al. (2015) disclose a method of quantification of MHC, in which cells are first treated and lysed with a nondenaturing detergent and MHC peptide complexes are then precipitated by applying the complex lysate to an affinity column coupled with monoclonal antibody (mAb) specific for a certain MHC class or allotype (Caron et al., 2015). This step of immunoprecipitation is error prone, as it sample material will get lost. This results in imprecise quantification.

Apps et al. (2015) have disclosed methods for the relative quantification of different HLA class I proteins in normal and HIV-infected cells. For this purpose, they have, inter alia, used digested immunoprecipitates from cultured B-LCL (B lymphoblastoid cells) or PBLs (Peripheral Blood Lymphocytes) freshly isolated from normal donors with trypsin and analyzed the digested and purified sample by liquid chromatography coupled to tandem mass spectrometry (LC-MS/MS) using an LTQ Orbitrap XL mass spectrometer (Thermo Fisher Scientific) (Apps et al., 2015).

To identify and relatively quantify the MHC subtypes HLA-A*02:01, HLA-B*44:02, HLA-C*05:01, and HLA-E, the authors used sets of between two and four peptides per MHC subtype. The sequences of these peptides corresponded to a stretch, domain, or epitope of each of one of HLA-A*02:01, HLA-B*44:02, HLA-C*05:01, and HLA-E (in total, they used eleven peptides for the entire set of four different HLAs). Both “heavy” isotope-labelled and “light” unlabelled peptide sets were used (Apps et al., 2015).

Heavy isotope-labelled peptides were spiked into the sample. To relatively quantify the different immunoprecipitated MHC proteins, a calibration curve was generated for each peptide by analyzing increasing amounts of synthetic “light” peptides mixed with a fixed amount of “heavy” peptide added to biological samples (Apps et al., 2015).

As a result, the authors were able to determine on freshly isolated PBLs from normal donors, that HLA-A and HLA-B proteins were expressed at similar levels relative to each other, but four to five times higher in relation to HLA-C. HLA-E was expressed at levels 25 times lower than HLA-C. On HIV-infected cells, HLA-A and HLA-B were reduced by a magnitude that varied between infected cultured cells (Apps et al., 2015).

However, the method of Apps et al. is not suitable to absolutely quantify MHC molecules in the sample, because
a) the sample to be analyzed has been obtained by immunoprecipitation, in which process part of the MHC proteome gets lost, and
b) the calibration curve used does not factor in MHC proteins, yet titrates increasing amounts of synthetic “light” peptides against a fixed amount of “heavy” peptides,

Further, the method of Apps et al. is also not suitable to universally quantify MHC molecules in different samples.

Also, Apps et al. does not consider the cell density count, so no absolute quantification is possible, as provided in a preferred embodiment of the present invention.

Still, the method of Apps et al. cannot be extended to other samples containing other HLA allotypes, hence is only applicable to the respective samples discloses therein.

Still, because, technically, the peptides were quantified, and not the entire HLA proteins, variations in the quantity of the respective peptides of one set representative for a given HLA subtype were accounted for by calculating the median of the different quantities. Because of the fact that each set contained only two to four peptides, such approach is relatively unreliable.

Hence, it is one other object of the present invention to provide means to predict a therapeutic window for one of the therapeutic modalities discussed above.

It is one other object of the present invention to provide means to determine whether or not a given MHC subtype is expressed in a sample of interest, to be able to assess whether or not a given the therapeutic modality can be used.

It is one other object of the present invention to provide means to enable the quantitative determination of at least one MHC subtype in a given sample.

### BRIEF DESCRIPTION OF THE DRAWINGS

FIG. 1 gives an overview over a workflow that is carried out according to one embodiment of the invention.

FIG. 2 shows the workflow of liquid chromatography coupled to tandem mass spectrometry (LC-MS/MS) used according to one embodiment of the invention. The sample is injected into the LC system (mostly HPLC, high performance LC) to partition the different peptides according to their size, and forward them to the mass spectrometer, where the peptides are ionized, accelerated, and analyzed by mass spectrometry (MS1). Ions from the MS1 spectra are then selectively fragmented and analyzed by a second stage of mass spectrometry (MS2) to generate the spectra for the ion fragments. While the diagram indicates separate mass analyzers (MS1 and MS2), some instruments utilize a single mass analyzer for both levels of MS.

FIG. 3 shows peptide fragments obtained from trypsin (in vitro or in silico) digestion of HLA-A*02:01. As discussed elsewhere herein, trypsin cleaves C-terminally of the amino acids K (Lys) and R (Arg). Peptides obtained in such way and eligible to be used for the internal standard are called “sample peptide analogues” (marked with SEQ ID NO. 1-10) herein.

In order to qualify as a sample peptide analogue to be used for the internal standard, the peptide should (i) not contain C (Cys), (ii) preferably not contain M (Met), although the latter can be replaced by methionine sulfoxide (MetO) (see SEQ ID NO 7), and (iii) should not comprise an N-glycosylation motif, such as NXS or NXT. For clarity purposes, M, C, and NXT/NXS are marked underlined.

FIG. 4 shows sample peptide analogues (also called peptides in this context) that can be used in an internal standard for a method according to the present invention. B stands for methionine sulfoxide, the asterisk shows optionally isotopically labelled amino acid residues. Note that, technically, also other residues in the peptides can be isotopically labelled, with the exception of Alanine and Glycine. Note that, in all sets of sample peptide analogues, peptides with overhangs can be replaced by the non-overhang counterparts and vice versa. E.g., instead of the peptide of SEQ ID NO 3, also the peptide of SEQ ID NO 20 can be used, or instead of the peptide of SEQ ID NO 30, also the peptide of SEQ ID NO 13 can be used

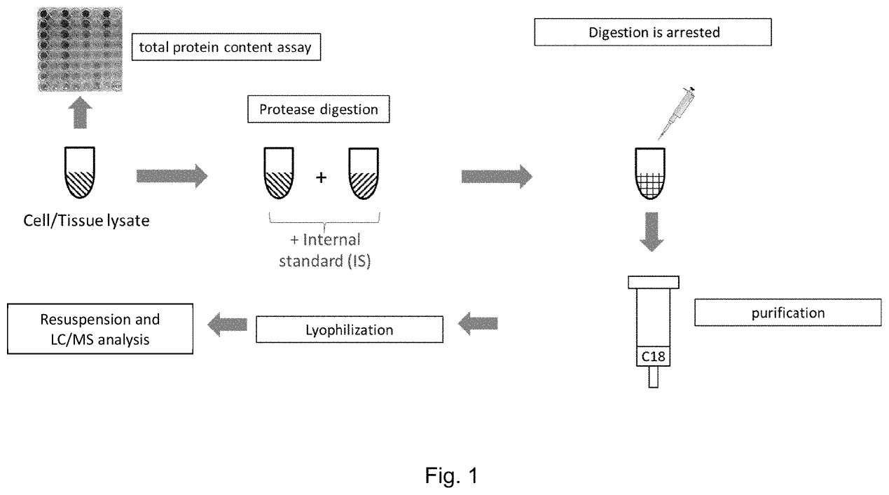

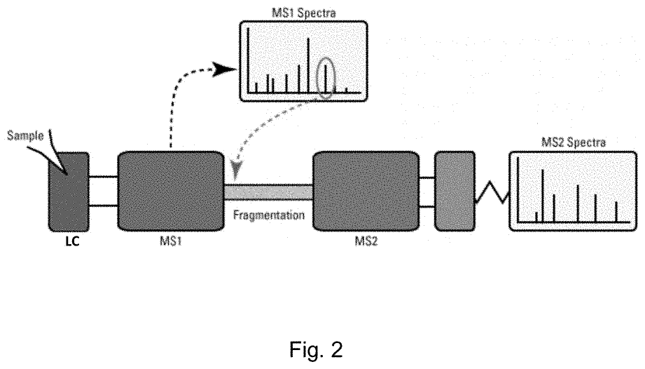

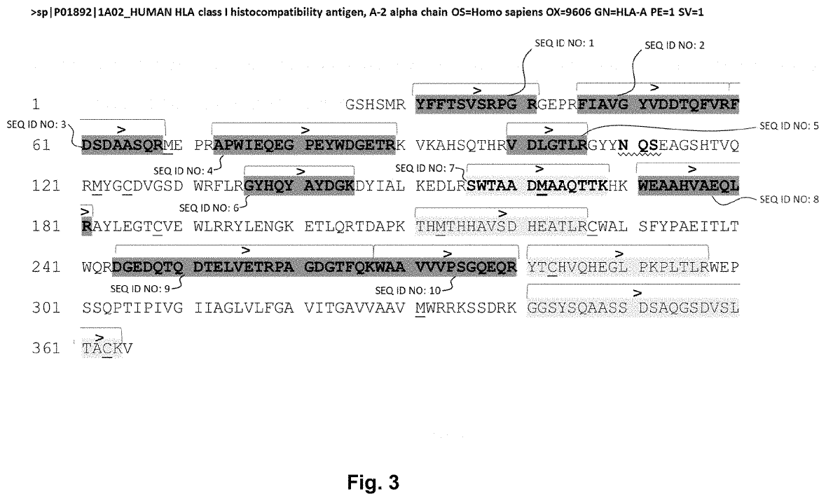

While the peptides of SEQ ID NO 1-10 (or their counterparts comprising overhangs, SEQ ID NO 18-27) and the SEQ ID NO 44-62 (or their counterparts comprising overhangs, SEQ ID NO 18-27) can be used to measure HLA-A*02:01; HLA-A*01:01; HLA-A*03:01; HLA-A*24:02; HLA-B*07:02; HLA-B*08:01; HLA-B*44:02 and/or HLA-B*44:03, the peptides of SEQ ID NO 11-12 (or their counterparts comprising overhangs, SEQ ID NO 28-29) can be used to measure 13-2 microglobulin, and the peptides of SEQ ID NO 13-17 (or their counterparts comprising overhangs, SEQ ID NO 30-34) can be used to measure at least one of H2A, histone H2B, or histone H4.

FIG. 5 shows an exemplary analysis step with LC-MS, and the subsequent MS/MS consisting of MS1 and MS2. A peptide taken from MS1 was fragmented by higher-energy collisional dissociation (HCD). Many copies of the same peptide (YLLPAIVHI) are fragmented at the peptide backbone to form a, b, and y ions. The spectrum consists of peaks at the m/z (mass to charge) values of the corresponding fragment ions.

FIG. 6 shows the principle of the internal standard method. A calibration curve is generated for each corresponding sample that is analyzed. For each sample to be analyzed a set of calibration samples is prepared comprising
(i) increasing concentrations of refolded monomer (MRF) comprising an MHC allotype (e.g., MHC A*02:01) and 132M,
(ii) internal standard in fixed concentration,
(iii) optionally, protein lysate, e.g. from yeast, which does not release any MHC sequence-identical peptides after tryptic digestions, as protein background.

The calibration sample is treated in the same manner as the actual sample, meaning in particular the digestion, and is subsequently subjected to the step of chromatography and/or spectrometry analysis. A calibration curve function is calculated from the ratio of MS signals by logistic regression.

FIG. 7 shows a hypothetical peptide-specific calibration curve along with its linear regression and corresponding equation.

FIGS. 8A-8D show absolute quantification of HLA-A*02:01 & 132m in human acute myeloid leukemia cell line MUTZ-3. (FIG. 8A) Quantification of respective peptides. Peptides unique to HLA-A*02:01 according to sample-specific typing are shown as squared bars. Those which also map to other HLA allotypes are shown as white bars. Underlying sample HLA typing with respective information is shown in (FIG. 8B). (FIG. 8C) Respective peptides are merged together and yield the corresponding protein concentration e.g. average of SEQ ID NO 4, 6 and 8 yields absolute abundance of HLA-A*02:01 in this example. (FIG. 8D) Factoring in the respective sample protein concentration, total cell lysate volume and the cell count translates the absolute protein concentration into the absolute quantity (number of molecules) per cell.

FIGS. 9A-9D show absolute quantification of HLA-A*02:01 & β2m in a human hepatocellular carcinoma sample. (FIG. 9A) Quantification of respective peptides. Peptides unique to HLA-A*02:01 according to sample-specific typing are shown as squared bars. Those which also map to other HLA allotypes are shown as white bars. Underlying sample HLA typing with respective information is shown in (FIG. 9B). (FIG. 9C) Respective peptides are merged together and yield the corresponding protein concentration e.g. average of SEQ ID NO 4, 5, 8 and 10 yields absolute abundance of HLA-A*02:01 in this example. (FIG. 9D) Factoring in the respective sample protein concentration, total cell lysate volume and the cell count translates the absolute protein concentration into the absolute quantity (number of molecules) per cell.

FIG. 10 shows the different sample peptide analogues (also called peptides in this context) that can be used in an internal standard for a method according to the present invention. Note that, in all sets of sample peptide analogues, peptides with overhangs can be replaced by the non-overhang counterparts and vice versa. E.g., instead of the peptide of SEQ ID NO 1, also the peptide of SEQ ID NO 18 can be used, or instead of the peptide of SEQ ID NO 26, also the peptide of SEQ ID NO 9 can be used.

Different sample peptide analogues can be used to quantify different HLA allotypes. In order to quantify, in a sample. More than one allotype, specific sets of sample peptide analogues can be selected based on this table. While some sample peptide analogues are exclusive for a given allotype, other represent more than one allotype. Still, because, in samples where different allotypes are present, the respective allotypes are unevenly distributed (with one in a significant majority over others) even those allotypes for which no “exclusive” sample peptide analogue exists can be quantified.

B stands for methionine sulfoxide, the asterisk shows optionally isotopically labelled amino acid residues. Note that, technically, also other residues in the peptides can be isotopically labelled, with the exception of Alanine and Glycine.

Of course, these sample peptide analogues can be combined with sample peptide analogues that allow measurement of ß-2 microglobulin. For example, the peptides of SEQ ID NO 11-12 (or their counterparts comprising overhangs, SEQ ID NO 28-29) can be used for this purpose.

Further, these sample peptide analogues can be combined with sample peptide analogues that allow measurement of at least one of H2A, histone H2B, or histone H4. For example, the peptides of SEQ ID NO 13-17 (or their counterparts comprising overhangs, SEQ ID NO 30-34) can be used for this purpose.

FIGS. 11A-11D shows absolute quantification of HLA-A*02:01, HLA-B*07:02 & β2m in human small cell carcinoma of the lung. (FIG. 11A) Quantification of respective peptides. Peptides unique to HLA-A*02:01 or HLA-B*07:02 according to sample-specific typing are either shown as large-squared or small-squared bars, respectively. Those which also map to other HLA allotypes are shown as white bars. Underlying sample HLA typing with respective information is shown in (FIG. 11B). (FIG. 11C) Respective peptides are merged together and yield the corresponding protein concentration e.g. average of SEQ ID NO 1, 4, 6 and 8 yields absolute abundance of HLA-A*02:01 in this example. (FIG. 11D) Factoring in the respective sample protein concentration, total cell lysate volume and the cell count translates the absolute protein concentration into the absolute quantity (number of molecules) per cell.

FIGS. 12A-12B show the calculated absolute cell count for selected samples from different tissue types. The respective cell count was derived via the sample-specific absolute histone abundance, as determined via LC-MS, and reversely correlated with the respective PBMC-based calibration curve (FIG. 12A) Absolute sample cell counts from either spleen, PBMCs, hepatocellular carcinoma (HCC), kidney, adipose tissue, heart and cartilage tissues are shown.

The cell count is independently calculated for all four selected histone peptides H2ATR-001, H2BTR-001, H4TR-001 & H4TR-002 and plotted as one bar, respectively. The median cell count derived from all four histones is plotted as a black bar per sample. The y scale depicts the absolute cell number per sample. (FIG. 12B) shows the median cell count per sample, as also shown in part (FIG. 12A) along with the respective protein concentration and absolute tissue weight per sample. For blood cells/PBMCs, the known manual cell count is plotted instead of the tissue weight.

FIG. 13 depicts the respective PBMC-based calibration curves to transform histone copies into an absolute cell number. The PBMC cell number (determined via manual cell counting) is shown on the x scale whereas the respective total histone count per PBMC sample, determined via LC-MS, is shown on the y scale. Per histone peptide (H2ATR-001, H2BTR-001, H4TR-001 & H4TR-002), one calibration curve exists. The fitted regression curve per histone peptide is shown as a dotted line.

### SUMMARY OF THE INVENTION

The invention and general advantages of its features will be discussed in detail below.

In the following, a first aspect of the present invention will be discussed, which relates to a novel and inventive method of determining the MHC content in a sample. Technology-wise, this method has large overlaps with a method according to a second aspect of the invention, in which the cell count in a sample is quantified. Therefore, preferred embodiments discussed in the context of the first aspect of the invention are deemed to be also disclosed with regard to the second aspect, and vice versa.

According to said first aspect of the invention, a method for the absolute quantification of one or more MHC molecules in a test sample comprising at least one cell is provided. The method comprises at least the steps of:
a) homogenizing the sample,
b) adding an internal standard to the sample,
c) digesting the homogenized sample with a protease, before or after addition of the internal standard,
d) subjecting the digested sample to a step of chromatography and/or spectrometry analysis, and
e) quantifying the one or more MHC molecules in the test sample

As used herein, the term “MHC molecule” refers to the major histocompatibility complex molecules. Such molecules are present on the cellular surface of most cells, where they display short peptides, which are molecular fragments of proteins. Presentation of pathogen-derived peptides, for example, results in the elimination of an infected cells cell by T cells of the immune system via T-cell receptors recognizing the specific peptide-MHC complex (pMHC).

As used herein the term “test sample” is meant to refer to a sample in which the one or more MHC molecules are to be quantified. Such test sample is for example a tissue sample, optionally a tumor tissue sample, a cell line (either primary cell line or immortalized). Further preferred embodiments of the test sample (also called “sample” herein) are disclosed elsewhere herein.

As used herein the term “calibration sample” is meant to refer to a sample comprising an MHC molecule standard at varying concentrations.

As used herein, the term “MHC molecule standard” is meant to refer to a HLA monomer. Such HLA monomer is a pHLA monomer, i.e., a HLA monomer to which a peptide is complexed. Optionally, the HLA monomer has been recombinantly produced. Optionally, the recombinantly produced HLA monomer is refolded.

As used herein the term “sample peptide analogues” is meant to refer to peptides that are added (“spiked”) to the test sample, and have identical or similar characteristics, (e.g., sequences) as the peptides that are obtained by protease digestion of the test sample.

In one embodiment, the sample obtained by the digestion is purified after step c) and prior to step d).

In some embodiments, the sample peptide analogues are isotopically labelled, as described elsewhere herein. In some embodiments, the sample peptide analogues comprise an overhang of amino acids at least N- or C-terminally, as described elsewhere herein, in such way that, after, protease digestion, the resulting digestion products are, in length and sequence, identical to the peptides that are obtained by protease digestion of the test sample.

In some embodiments, these sample peptide analogues are comprised in what is called the internal standard

As used herein the term “query proteins” is meant to refer to the proteins the quantity of which is to be determined. This relates, for example, to (i) beta-2-microglobulin (β2m), (ii) the MHC proteins (like the different HAL allotypes), and (iii) the protein the abundance of which is roughly proportional to the total number of cells in the sample (like e.g. the histones).

Contrary to the method of Apps et al. and Caron et al, the method according to this embodiment is actually suitable to absolutely quantify MHC molecules in the sample, because the sample to be analyzed has not been obtained by immunoprecipitation (in which process part of the MHC proteome gets lost), yet is processed directly. Further advantages relative to the method of Apps et al., are disclosed elsewhere herein.

According to one embodiment, the protease used for digesting the sample is trypsin. Trypsin has some properties that make it specifically suitable for the method of the present invention. Its cleavage motives are shown in the following table, with the arrow indicating the cleavage site:

↓

N′-[...]-X₁-K/R-X₂-[...]-C′
N′-[...]-W--K---P-[...]-C′
N′-[...]-M--R---P-[...]-C′

where X₂ cannot be P (Pro). Because of the relative simplicity of the cleavage site, trypsin creates relatively short fragments, which, because the cleavage site comprises a charged amino acid (either K (Lys) or R (Arg)), have a relatively constant mass-to-charge ratio.

In mass spectrometry, a constant mass-to-charge ratio (symbols: m/z, m/e) is highly advantageous to ensure that the resolution of the spectrum is not affected by charge-induced artifacts.

According to one embodiment, the protease, in particular the trypsin, is immobilized on a matrix, e.g. on specific beads. In such way, the protease can be removed from the sample prior to further processing.

A commercially available kit that is suitable for the above purpose is the SMART Digest™ kit (Thermo Scientific™). This kit comprises porcine trypsin immobilized on particular beads.

According to one embodiment, the digestion takes place at a temperature of between ≥45 and ≤75° C. According to one embodiment, the digestion takes place in a planar orbital shaker at a speed of between ≥1000 and ≤2000 rpm. According to one embodiment, the digestion is carried for a period of between ≥80 and ≤120 min

In one specific embodiment, the digestion takes place at a temperature of 70° C. in a planar orbital shaker at a speed of between 1400 rpm for 105 min.

According to one embodiment, the method further comprises the step of determining the total protein concentration in the sample prior to digestion.

According to one embodiment, the total protein concentration in the sample is determined by a bicinchoninic acid assay (BCA assay). The BCA assay primarily relies on two reactions. First, the peptide bonds in the protein(s) reduce Cu²⁺ ions from the copper(II) sulfate to Cu⁺ (a temperature-dependent reaction). The amount of Cu²⁺ reduced is proportional to the amount of protein present in the solution. Next, two molecules of bicinchoninic acid chelate with one Cu⁺ ion, forming a purple-colored complex that strongly absorbs light at a wavelength of 562 nm.

The amount of protein present in a solution can be quantified by measuring the absorption spectra and comparison with protein solutions of known concentration.

Details of the method are disclosed in Olson and Markwell, the content of which is incorporated herein by reference for enablement purposes (Olson and Markwell, 2007).

According to one embodiment of the method according to the invention, prior or after homogenization, the sample is not treated with, or obtained by, immunoprecipitation.

According to one embodiment of the method according to the invention, the sample is selected from the group consisting of
an extract of a biological sample comprising proteins
a primary, non-cultured sample, and/or
sample obtained from one or more cell lines.

According to one embodiment, the primary, non-cultured sample is selected from the group consisting of a tissue sample, a blood sample, a tumor sample, or a sample of an infected tissue.

According to one embodiment, the primary, non-cultured sample is a piece of tissue. According to one embodiment, the primary, non-cultured sample is a biopsy. According to one embodiment, the primary, non-cultured is a smear sample. According to one embodiment, the primary, non-cultured sample is a fine-needle aspiration (FNA), or sampling (FNS)

According to one embodiment, the primary, non-cultured sample is a fresh sample. According to one embodiment, the primary, non-cultured sample is a frozen sample. In still one embodiment, the primary, non-cultured sample is an otherwise preserved samples, like e.g. an embedded or frozen sample (e.g. FFPE-preserved sample, Bambanker™-preserved frozen sample).

According to one embodiment, the cell line is a cell line derived from a tumor. In another embodiment, this cell line could be further passaged in vitro (e.g. cell culture) or in vivo (e.g. mouse xenograft). In another embodiment, the cell line is an immortalized cell line derived from human tissue. In still one embodiment, the cell line is a stem cell line.

According to one embodiment of the method according to the invention, the primary sample is selected from the group consisting of a tissue sample, a blood sample, a tumor sample, or a sample of an infected tissue.

According to one embodiment of the method according to the invention, the MHC is MHC class I (MHC-I).

According to one embodiment of the method according to the invention, the MHC is a human MHC protein, preferably human leukocyte antigen A (HLA-A) and/or human leukocyte antigen B (HLA-B).

In further embodiments, the MHC is human leukocyte antigen C (HLA-C) and/or human leukocyte antigen E (HLA-E).

This involves different HLA allotypes, including also mixtures of different HLA allotypes.

According to one embodiment of the method according to the invention, the HLA allotype is HLA-A*02.

According to one embodiment of the method according to the invention, the MHC is at least one HLA allotype selected from the group consisting of HLA-A*02:01; HLA-A*01:01; HLA-A*03:01; HLA-A*24:02; HLA-B*07:02; HLA-B*08:01; HLA-B*44:02 and/or HLA-B*44:03.

According to another embodiment of the method according to the invention the HLA-A is HLA-A*02:01.

The peptides shown in Table 1 hereinbelow are particularly suitable for the quantification of HLA-A*02:01.

The peptides shown in Table 4 hereinbelow are particularly suitable for the quantification of at least one of HLA-A*01:01; HLA-A*03:01; HLA-A*24:02; HLA-B*07:02; HLA-B*08:01; HLA-B*44:02 and/or HLA-B*44:03.

According to one embodiment of the method according to the invention, after digestion, the sample is treated with a strong acid to interrupt the digestion and/or precipitate or denaturate or inactivate the protease.

According to one embodiment, trifluoroacetic acid (TFA) is used for this purpose, added to the sample to arrive at a concentration of between ≥0.05 and ≤5% v/v.

By adding such acid, the resulting pH shift inactivates e.g. trypsin, which has a pH optimum of between pH 7 and 8.

According to one embodiment of the method according to the invention, purifying the sample obtained by the digestion comprises solid-phase extraction. In such approach, a C18 resin is optionally used.

Solid-phase extraction (SPE) is an extractive technique by which compounds that are dissolved or suspended in a liquid mixture are separated from other compounds in the mixture according to their physical and chemical properties. Analytical laboratories use SPE to concentrate and purify samples for analysis. SPE can be used to isolate analytes of interest from a wide variety of matrices, including urine, blood, water, beverages, soil, and animal tissue.

SPE uses the affinity of solutes dissolved or suspended in a liquid (known as the mobile phase) for a solid through which the sample is passed (known as the stationary phase) to separate a mixture into desired and undesired components. The result is that either the desired analytes of interest or undesired impurities in the sample are retained on the stationary phase. The portion that passes through the stationary phase is collected or discarded, depending on whether it contains the desired analytes or undesired impurities. If the portion retained on the stationary phase includes the desired analytes, they can then be removed from the stationary phase for collection in an additional step, in which the stationary phase is rinsed with an appropriate eluent.

Many of the adsorbents/materials are the same as in chromatographic methods, but SPE is distinctive, with aims separate from chromatography, and so has a unique niche in modern chemical science.

According to one embodiment, the solid-phase extraction uses octadecyl silica to retain non-polar compounds by strong hydrophobic interaction. This approach is also called C18 SPE.

A commercially available tool that is suitable for the above purpose are the Thermo Scientific™ SOLAμ™ Solid Phase Extraction (SPE) plates.

According to one embodiment, SPE may be used to remove impurities, such as salts and high-molecular weight compounds, e.g., trypsin beads (see examples 1 and 2).

According to one embodiment of the method according to the invention, after purifying the sample the resulting purification product is dried, preferably by lyophilization.

According to one embodiment, after drying the purification product, the purification product is re-suspended. According to one embodiment, the re-suspension takes place in aqueous formic acid (FA). The concentration thereof is in the range of 1-10%. In one specific embodiment, the concentration is 5%.

According to one embodiment of the method according to the invention, the step of chromatography and/or spectrometry analysis comprises LC-MS/MS analysis.

The term “LC-MS/MS”, as used herein, includes two process steps, namely
a) Liquid chromatography (mostly HPLC), and
b) Tandem mass spectrometry, also known as MS/MS or MS2

The combination of liquid chromatography (mostly HPLC) and tandem mass spectrometry is extremely helpful in sophisticated protein or peptide analysis. The method combines the physical separation capabilities of liquid chromatography (or HPLC) with the mass analysis capabilities of a mass spectrometer (MS).

The liquid chromatography separates the peptide sample according to the molecular mass or size and/or the degree of hydrophobicity of the comprised peptides. Via an interface, the separated components are transferred from the LC column into the MS ion source. The mass spectrometry provides compositional identity (e.g. amino acid sequence) of the individual components with high molecular specificity and detection sensitivity

Mass spectrometry is a sensitive technique used to detect, identify, and quantitate molecules based on their mass-to-charge ratio (m/z).

The development of macromolecule ionization methods, including electrospray ionization (ESI) and atmospheric pressure chemical ionization (APCI), enabled the study of protein structures by MS. Mass spectrometry measures the m/z ratio of ions to identify and quantify molecules in simple and complex mixtures.

MS/MS is a technique where two or more mass analyzers are coupled together using an additional reaction step to increase their abilities to analyze the chemical composition of samples.

In peptide analysis, the peptide molecules of the sample are ionized and the first analyzer (designated MS1) separates these ions by their mass-to-charge ratio (often given as m/z or m/Q). Ions of a particular m/z-ratio coming from MS1 are selected and then made to split into smaller fragment ions, e.g. by collision-induced dissociation (CID), higher-energy collisional dissociation (HCD) or electron-transfer dissociation (ECD). Three different types of backbone bonds in peptides are thus broken to form peptide fragments: alkyl carbonyl (CHR—CO), peptide amide bond (CO—NH), and amino alkyl bond (NH—CHR).

These fragments are then introduced into the second mass analyzer (MS2), which in turn separates the fragments by their m/z-ratio and detects them. The fragmentation step makes it possible to identify and separate precursor ions that have very similar m/z-ratios in regular MS1 mass analyzers.

Tandem mass spectrometry can produce a peptide sequence tag that can be used to identify a peptide in a protein database. A notation has been developed for indicating peptide fragments that arise from a tandem mass spectrum. Peptide fragment ions are indicated by a, b, or c if the charge is retained on the N-terminus and by x, y, or z if the charge is maintained on the C-terminus. The subscript indicates the number of amino acid residues in the fragment.

Respective methods of LC-MS/MS-based proteomics applications are disclosed, inter alia, in U.S. Pat. No. 9,343,278B2, the content of which is enclosed herein for enablement purposes.

According to one embodiment of the method according to the invention, the step of chromatography and/or spectrometry analysis comprises sequencing at least one the peptides in the sample by de novo peptide sequencing.

In de novo peptide sequencing, the mass difference between two fragment ions is used to calculate the mass of an amino acid residue on the peptide backbone. The mass can uniquely determine the residue. For example, as shown in FIG. 7 the mass difference between the y₇ and y₆ ions is equal to 113 Da, which is the molecular mass of the amino acid residue L (Leu). Said process is continued until all the residues are determined. A mass table of amino acids is provided in Table 6.

TABLE 6

| | 3-letter | 1-letter | Average |
|---|---|---|---|
| Name | code | code | mass (Da) |
| Alanine | Ala | A | 71.08 |
| Arginine | Arg | R | 156.2 |
| Asparagine | Asn | N | 114.1 |
| Aspartic Acid | Asp | D | 115.1 |
| Cysteine | Cys | C | 103.1 |
| Glutamic Acid | Glu | E | 129.1 |
| Glutamine | Gln | Q | 128.1 |
| Glycine | Gly | G | 57.05 |
| Histidine | His | H | 137.1 |
| Isoleucine | Ile | I | 113.2 |
| Leucine | Leu | L | 113.2 |
| Lysine | Lys | K | 128.2 |
| Methionine | Met | M | 131.2 |
| Phenylalanine | Phe | F | 147.2 |
| Proline | Pro | P | 97.12 |
| Serine | Ser | S | 87.08 |
| Threonine | Thr | T | 101.1 |
| Tryptophan | Trp | W | 186.2 |
| Tyrosine | Tyr | Y | 163.2 |
| Valine | Val | V | 99.13 |

Respective algorithms and methods of MS-based de novo sequencing are disclosed, inter alia, in US20190018019A1, the content of which is enclosed herein for enablement purposes.

While mass spectrometry is extremely powerful when it comes to the determination of molecular masses, it is intrinsically not suitable for the quantification of the detected molecules.

The internal standard is added to the sample prior to the step of chromatography and/or spectrometry analysis. The process is called “spiking” herein, and the respective volume of internal standard that is added to the sample is called “spike”.

The molecules comprised in the internal standard in a defined concentration are also called “sample molecule analogues”, as they are chosen to reflect, in their elution and fragmentation properties, the peptides derived from digestion of molecules in the sample that are to be quantified.

The amounts/concentrations of the molecules comprised in a defined concentration can be readily adjusted and depend at least in part on the sample to be spiked and the method used for the analysis.

According to one embodiment of the method according to the invention, the internal standard comprises at least one peptide in a defined concentration.

The one or more peptides in the internal standard—also called “sample peptide analogues”—co-elute simultaneously with the peptides from the sample and are analyzed by MS and MS/MS simultaneously.

According to one embodiment of the method according to the invention, the internal standard comprises a set of three or more peptides—also called “sample peptide analogues”, wherein the sequence of each peptide corresponds to a stretch, domain, or epitope of one HLA allotype selected from the group consisting of human leukocyte antigen A (HLA-A) and/or human leukocyte antigen B (HLA-B).

In further embodiments, the sequence of each peptide corresponds to a stretch, domain, or epitope of one HLA allotype selected from the group consisting human leukocyte antigen C (HLA-C) and/or human leukocyte antigen E (HLA-E).

According to one embodiment of the method according to the invention, the MHC is MHC class I (MHC-I).

This means that, in such embodiment, at least five peptides are being used per HLA allotype. The inventors have found that this minimum ensures reliable and reproducible quantification of all members of the respective HLA allotype.

According to one embodiment of the method according to the invention, the HLA to a stretch, domain, or epitope of which the sequences of the peptides correspond is HLA-A*02.

According to one embodiment of the method according to the invention, the HLA to a stretch, domain, or epitope of which the sequences of the peptides correspond is at least one selected from the group consisting of HLA-A*02:01; HLA-A*01:01; HLA-A*03:01; HLA-A*24:02; HLA-B*07:02; HLA-B*08:01; HLA-B*44:02 and/or HLA-B*44:03.

According to one embodiment of the method according to the invention, the HLA to a stretch, domain, or epitope of which the sequences of the peptides correspond is HLA-A*02:01.

As used herein, the term HLA genotype refers to the complete set of inherited HLA genes.

As used herein the term HLA allele refers to alternative forms of an HLA gene found in the same locus in different individuals. Due to the high degree of polymorphisms of HLA genes in the human population the number of alleles is extremely high. In December 2020, the IPD-IMGT/HLA Database (release 3.42.0, 2020-10-15) comprised a total of 6,291 HLA-A alleles (3,896 allotypes), 7,562 HLA-B alleles (4,803 allotypes), and 6,223 HLA-C alleles (3,618 allotypes) (Robinson et al., 2015).

As used herein, the term HLA allotype refers to the different HLA protein forms encoded by respective HLA alleles. Due to the degenerate genetic code different HLA alleles can encode for the same HLA allotype.

As used herein, the term HLA haplotype refers to the set of HLA alleles contributed by one parent which are encoded together on one chromosome.

HLA-A*02:01 (Uniprot ID P01892) is an allotype of the HLA allele HLA-A*02, within the HLA-A gene group. HLA-A*02 is one particular class I major histocompatibility complex (MHC) allele group at the HLA-A locus. The HLA-A*02 allele group comprises 1,454 alleles encoding for a somewhat lower number of different proteins (allotypes; IPD-IMGT/HLA Database release 3.42.0, 2020-10-15) (Robinson et al., 2015).HLA-A*02 is globally common, but particular variants of can be separated by geographic prominence. HLA-A*02:01 has the highest frequency worldwide, with e.g. 26.7% in a German study group including 39,689 individuals (Allele Frequency Net Database; Germany pop 8; n=39,689; (Gonzalez-Galarza et al., 2015)).

According to one embodiment, the set comprises at least two peptides having a sequence which corresponds to a stretch, domain, or epitope of at least two different HLA allotypes. In this embodiment, the method enables quantification of a further HLA allotype.

According to one embodiment, a further set of three or more peptides—also called “sample peptide analogues”—is used whose sequences correspond to a stretch, domain, or epitope of such further HLA allotype.

According to one embodiment of the method according to the invention, the at least one peptide in the internal standard comprises an overhang of amino acids at the N-terminus and/or at the C-terminus, wherein the overhang of amino acids comprises a protease cleavage site.

Said protease cleavage site is, in one embodiment, a trypsin cleavage site, as disclosed elsewhere herein.

On that basis, whenever, in the present specification, a peptide is referred to without such overhangs (e.g., SEQ ID NOs 1-17, or 44-62), the respective peptide with overhangs is likewise deemed to be referred to (e.g., SEQ ID NOs 18-34, or 63-81. This means that sets of peptides with and peptides without such overhangs can be used and are disclosed herein.

As used herein, the term “overhang of amino acids” means that the peptides are selected in such way that they comprise one or more further amino acid residues beyond at least the C- or N-terminal cleavage site of the protease that has been used for the template protein digestion.

According to one embodiment, said overhang is present both N- and C-terminally. According to one embodiment, each of said overhangs can have a length of between ≥1 AA and ≤10 AA residues. It should be noted that in the overhangs, C or M residues can be present.

In all these cases, the peptides of the internal standard, or the internal standard as a whole, is/are subjected to protease digestion under identical conditions as the sample, in particular with the same protease.

See the following table for two examples, with the overhangs having an exemplary length of 3 AA residues being shown in italics underline (in this case, the protease is trypsin):

| | |
|---|---|
| Seq. of | [Xn] |
| template | PLVEEPQNLI [symbol] QNCELFEQLGEY [symbol] FQNALLV |
| protein | [Xn] |
| Peptide for IS | LIKQNCELFEQLGEYKFQN |
| Seq. of | [Xn] |
| template | TLFGD [symbol] LCTVATL [symbol] ETYGE |
| protein | [Xn] |
| Peptide for IS | GDKLCTVATLRETY |

Using peptides with overhangs for the internal standard, when the latter is added to the sample prior to digestion, makes sure that the peptides of the standard, which also subjected to protease digestion, just as the test sample itself, also undergo protease cleavage. Without the overhangs, the peptides would be unaffected by the protease treatment. This helps to better mimic digestion efficiency of the process, and make sure that the peptides of the internal standard faithfully reflect the composition of the peptides of the sample as achieved after the protease digestion.

According to one embodiment of the method according to the invention, the set of peptides further comprises at least one peptide the sequence of which corresponds to a stretch, domain, or epitope of beta-2-microglobulin (β2m).

β2m (Uniprot ID P61769) is part of the heterodimeric MHC class I complex, provides stability thereto complex and participates in the recognition of peptide-MHC class I complex by CD8 co-receptor. The peptide is non-covalently bound to the α subunit, it is held by the several pockets on the floor of the peptide-binding groove. β2m lies next to the α₃ chain of HLA on the cell surface. Unlike α₃, β2m has yet no transmembrane region.

Interestingly, while different alleles (genotypes) and proteins (allotypes) of HLA exist, no such variants of β2m exists. In other words: All different HLA allotypes comprise, or form a complex with, the same β2m molecule. Hence, quantification of β2m in the sample can be used to quantify the entirety of all HLA class I allotypes in a sample.

In such way, the method allows to quantify the share of specific HLA allotypes, like, e.g., HLA-A*02:01 within the entirety of HLA class I molecules in a sample.

According to one embodiment of the invention, the method further comprises the step of determining the total cell count in the sample.

Depending on the exact sample type, the cell count can be determined by different approaches. In case of cultured cells (i.e. cell line samples), the cell count can previously be determined microscopically, and can then be factored in.

Another option for estimating sample-specific cell count is to reversely correlate its tissue weight with the cell count. This is achieved via a tissue weight-based regression curve correlated with a cohort of data, for which cell counts have been previously determined via fluorescence-based DNA quantification.

As another option for primary, non-cultured samples (e.g., tissues or blood), the cell count can be determined by determining the concentration of a peptide the sequence of which corresponds to a stretch, domain, or epitope of one or more proteins the abundance of which is roughly proportional to the total number of cells in the sample.

According to one embodiment of the method according to the invention, the set of peptides further comprises at least one peptide the sequence of which corresponds to a stretch, domain, or epitope of one or more proteins the abundance of which is roughly proportional to the total number of cells in the sample.

The term a “protein the abundance of which is roughly proportional to the total number of cells in the sample” relates to a protein the concentration of which per cell is roughly constant.

This condition applies, e.g., to histones. Histones are highly basic proteins found in eukaryotic cell nuclei that pack and order the DNA into structural units called nucleosomes. Histones are the chief protein components of chromatin, acting as spools around which DNA winds, and playing a role in gene regulation. Because, in a diploid cell, the amount of DNA is constant, the amount of histone is also constant Five major families of histones exist: H1/H5, H2A, H2B, H3, and H4. Histones H2A, H2B, H3, and H4 are known as the core histones, while histones H1/H5 are known as the linker histones.

According to one embodiment of the method according to the invention, at least one protein the abundance of which is roughly proportional to the total number of cells in the sample is a histone, e.g., Histone H2A, histone H2B, or histone H4.

Histone H2A (UniProt ID B2R5B3) is one of the main histone proteins involved in the structure of chromatin in eukaryotic cells. H2A utilizes a protein fold known as the “histone fold”. The histone fold is a three-helix core domain that is connected by two loops. This connection forms a “handshake arrangement”. Most notably, this is termed the helix-turn-helix motif, which allows for dimerization with H2B.

Histone H2B (UniProt ID B4DR52) is another one of the main histone proteins involved in the structure of chromatin in eukaryotic cells. Two copies of histone H2B come together with two copies each of histone H2A, histone H3, and histone H4 to form the octamer core of the nucleosome[2] to give structure to DNA.

Histone H4 (UniProt ID Q6B823) is yet another one of the main histone proteins involved in the structure of chromatin in eukaryotic cells. Histone proteins H3 and H4 bind to form a H3-H4 dimer, two of these H3-H4 dimers combine to form a tetramer. This tetramer further combines with two H2a-H2b dimers to form the compact Histone octamer core.

Generally, the abundance of histones, is due to their DNA-binding capacity, proportional to the total number of cells in the sample. Quantifying histones in a sample hence provides an estimate of the total number of cells comprised therein.

For this purpose, according to one embodiment, a calibration curve is established by titration of one or more cells vs. a histone-based signal, as obtained by the spectrometry methods disclosed herein. More precisely, the ratio of endogenous histone peptides obtained by tryptic digestion versus their heavy isotope-labelled internal standard peptides is determined.

In one example, the internal standard (IS) comprises a set of peptides the sequence of which corresponds to a stretch, domain, or epitope of the following proteins, as shown in the following table:

| | | Number of |
|---|---|---|
| | | different |
| Template protein | example | peptides in IS |
| beta-2-micro- | | ≥1-≤4 |
| globulin | | |
| HLA | HLA-A*02:01 | ≥5-≤20 |
| protein which | Histone, e.g., | ≥5-≤10 |
| is roughly | at least one | |
| proportional | of Histone H2A, | |
| to the total | Histone H2B, | |
| number of cells | and Histone H4 | |

Optionally, the set can comprise one or more further sets of ≥5-≤20 further peptides the sequence of which corresponds to a stretch, domain, or epitope of another HLA allotype different to HLA-A*02:01. In such way, more than one HLA allotype can be quantified.

In addition or as an alternative to determine the cell count in the sample, one can also determine the DNA content in the sample, as e.g. disclosed in (McCaffrey et al., 1988) McCaffrey et al (1988).

According to one embodiment, the sequence of at least one of the peptides of the internal standard which matches to one of the query proteins has been derived from the template protein by in silico protease digestion.

In silico protease digestion, as used herein, means that the template protein is analysed for potential protease cleavage sites, and the peptide sequences are then chosen according to the protein fragments that would have been created by the protease activity.

For example, as discussed above, trypsin cleaves C-terminally of K and R residues. Hence, an analysis of the template protein for potential trypsin cleavage sites delivers protein fragments that would have been created by the protease activity, which C terminally either have a K or R.

See the following table for two examples (with the sequence of the template protein chosen, for exemplary purposes only, from human serum albumin, K and R bold and underlined, and X being any proteinogenic amino acid (in this case, the protease is trypsin):

| | |
|---|---|
| Seq. of | [Xn] |
| template | PLVEEPQNLI [symbol] QNCELFEQLGEY [symbol] FQNALLV |
| protein | Xn] |
| Peptide for IS | QNCELFEQLGEYK |
| Seq. of | [Xn] |
| template | TLFGD [symbol] LCTVATL [symbol] ETYGE |
| protein | [Xn] |
| Peptide for IS | LCTVATL [symbol] |

According to one embodiment, at least one peptide of the internal standard is selected in such way that it does not comprise C residues.

C (Cys) comprises a thiol group which has the potential to build disulphide bridges with other cysteines in the same or other peptides. Hence, having cysteine comprising peptides in the internal standard could lead to artifacts caused by the formation of heterooligomers, and hence errors in the analysis.

According to one embodiment, at least one peptide of the internal standard is selected in such way that it does not comprise M residues. M (Met) comprises a thioether, and partly oxidizes during sample preparation, which hence leads to the generation of two different peptides (reduced M and oxidized M oxidized), both of which would have to be quantified.

As an alternative, M is replaced by methionine sulfoxide (MetO), for which the one letter code “B” is used herein.

According to one embodiment, at least one peptide of the internal standard is selected in such way that it does not comprise post-translational modifications.

This applies, inter alia, to as N-glycosylation. N-glycosylation motifs are NXS and NXT, so in this embodiment, care is taken that the peptides used for the internal standard do not comprise any of these motifs.

Other post-translational modifications that can preferably be avoided by respective selection of the peptides used for the internal standard (and avoidance of amino acid residues that are likely subject of such post-translational modifications) include, but are not limited to
mono, di- or trimethylation of e.g., lysine or arginine,
acetylation of e.g. lysine or asparagine, or
phosphorylation of e.g. tyrosine, threonine or serine.

According to one embodiment, the peptides of the internal standard are produced synthetically.

According to one embodiment, the peptides of the internal standard have a length, not including the overhangs, of between ≥4 and ≤50 AA. According to one embodiment, the peptides of the internal standard have a molecular weight, not including the overhangs, of between ≥400 and ≤5000 Da.

Of course, the length or weight of the peptides of the internal standard is also dictated by the cleavage characteristics of the protease, with some proteases creating, in general, larger fragments, and other creating shorter fragments.

With regard to the peptides of the internal standard, reference is further made to preferred embodiments and restrictions disclosed elsewhere herein in the context of the claimed set of peptides. Advantages and characteristics of these embodiments will not be repeated here to avoid lengthiness.

In the following, (i) beta-2-microglobulin (β2m), (ii) the HLA allotype, and (iii) the protein the abundance of which is roughly proportional to the total number of cells in the sample will also be called “query proteins” to which the peptides in the internal standard match.

According to one embodiment of the method according to the invention, the internal standard is added to the sample prior to the step of digesting the homogenized sample with a protease.

According to one embodiment of the method according to the invention, at least one molecule in the internal standard is labelled.

According to one embodiment, the label is at least one of
a metal-coded tag, and/or
an isotope label

Metal-coded tags (MeCAT) are based on chemical labeling, but rather than using stable isotopes, different lanthanide ions in macrocyclic complexes are used. The quantitative information comes from inductively coupled plasma MS measurements of the labelled peptides. MeCAT can be used in combination with elemental mass spectrometry ICP-MS allowing first-time absolute quantification of the metal bound by MeCAT reagent to a protein or biomolecule.

Thus, it is possible to determine the absolute amount of protein down to attomol range using external calibration by metal standard solution. It is compatible with protein separation by 2D electrophoresis and chromatography in multiplex experiments.

Labeling the molecules with isotope labels allows the mass spectrometer to distinguish, e.g., between identical proteins in separate samples.

One type of label, isotopic tags, consists of stable isotopes incorporated into protein crosslinkers that causes a known mass shift of the labelled protein or peptide in the mass spectrum. Differentially labelled samples are combined and analyzed together, and the differences in the peak intensities of the isotope pairs accurately reflect difference in the abundance of the corresponding proteins.

Another approach is the use of isotopic peptides. This approach entails spiking known concentrations of synthetic, heavy isotopologues of target peptides into the experimental sample and then performing LC-MS/MS. Peptides of equal chemistry co-elute from the LC and are analyzed by MS simultaneously. However, the abundance of the target peptide in the experimental sample is compared to that of its isotopologue and back-calculated to the initial concentration of the standard

According to one embodiment of the method according to the invention, one amino acid in at least one peptide in the internal standard is isotopically labelled by incorporation of ¹³C and/or ¹⁵N during synthesis.

According to one embodiment, only one amino acid in each peptide is labelled in such way. For that purpose, for each peptide, the amino acid residue that is to be labelled must be unique in said peptide. Such labelling supports successful discrimination between the endogenous peptides from the sample and the peptides from the internal standard. Generally, the mass shift caused by the isotopic labelling should create a minimal mass shift of 6 Da of the incorporated amino acid for peptides below 2,000 Da, to avoid overhang between the isotopic envelopes. In case of peptides larger than 2,000 Da, a labelled amino acid should be chosen which provides a minimal mass shift of 10 Da, such as labelled F (Phe) or Y (Tyr) to avoid isotope envelope overhangs.

See the following table for typical examples of labelled amino acids, and the resulting mass shift

| | | Molecular formula | Mass shift |
|---|---|---|---|
| AA | AA | of ¹³C/¹⁵N | relative |
| (Single | (Three | universally- | to |
| Letter | Letter | labelled free | unlabeled |
| Code) | Code) | amino acid | (Da) |
| A | ala | (¹³C)₃H₇(¹⁵N)O₂ | (+4) |
| R | arg | (¹³C)₆H₁₄(¹⁵N)₄O₂ | (+10) |
| N | asn | (¹³C)₄H₈(¹⁵N)₂O₃ | (+6) |
| D | asp | (¹³C)₄H₇(¹⁵N)O₄ | (+5) |
| C | cys | (¹³C)₃H₇(¹⁵N)O₂S | (+4) |
| Q | gln | (¹³C)₅H₁₀(¹⁵N)₂O₃ | (+7) |
| E | glu | (¹³C)₅H₉(¹⁵N)O₄ | (+6) |
| G | gly | (¹³C)₂H5(¹⁵N)O₂ | (+3) |
| I | ile | (¹³C)₆H₁₃(¹⁵N)O₂ | (+7) |
| L | leu | (¹³C)₆H¹³(¹⁵N)O₂ | (+7) |
| K | lys | (¹³C)₆H₁₄(¹⁵N)₂O₂ | (+8) |
| M | met | (¹³C)₅H₁₁(¹⁵N)O₂S | (+6) |
| F | phe | (¹³C)₉H₁₁(¹⁵N)O₂ | (+10) |
| P | pro | (¹³C)₅H₉(¹⁵N)O₂ | (+6) |
| S | ser | (¹³C)₃H₇(¹⁵N)O₃ | (+4) |
| T | thr | (¹³C)₄H₉(¹⁵N)O₃ | (+5) |
| Y | tyr | (¹³C)₉H₁₁(¹⁵N)O₃ | (+10) |
| V | val | (¹³C)₅H₁₁(¹⁵N)O₂ | (+6) |

According to one embodiment of the method according to the invention, a calibration routine is established, comprising the steps of
providing at least two calibration samples, the samples comprising an MHC molecule standard at varying concentrations and, added thereto, internal standard at a fixed concentration,
digesting the calibration sample with a protease, before or after addition of the internal standard
purifying the calibration sample obtained by the digestion,
subjecting the digested sample to a step of chromatography and/or spectrometry analysis

According to one embodiment of the method according to the invention,
a) the MHC molecule standard is a HLA monomer, and/or
b) the calibration samples further comprise yeast protein lysate

In one embodiment, the HLA monomer is a pHLA monomer, i.e., a HLA monomer to which a peptide is complexed. In one embodiment, the HLA monomer has been recombinantly produced. In one embodiment, the recombinantly produced HLA monomer is refolded.

Refolding of HLA monomers is for example disclosed in Garboczi et al. (1992), the content of which is incorporated herein by reference for enablement purposes.

The yeast protein lysate serves as a protein background to mimic the protein composition of the test samples. The inventors have verified that yeast protein lysate does not release any MHC sequence-identical peptides after tryptic digestion.

The HLA monomer (also called MRF herein, wherein R stands for “refolded”) contains within its primary structure all relevant peptide sequences that are comprised in the internal standard as peptide stretches.

According to one embodiment, the HLA monomer that is used as MHC molecule standard is a refolded pHLA-A*02:01 monomer.

Hence, in the calibration samples, the internal standard is kept constant and the concentration of the refolded HLA monomer is varied. In such manner, the HLA monomer that is used as MHC molecule standard serves as a titrated standard for quantification.

Contrary to the method of Apps et al., the method according to this embodiment is actually suitable to absolutely quantify MHC molecules in the sample, because the calibration curve used does indeed factor in MHC proteins, and does not merely titrate increasing amounts of synthetic “light” peptides against a fixed amount of “heavy” peptides.

The following table gives an example for such collection of calibration samples:

| | Total MRF | Internal |
|---|---|---|
| Calibration | concentration | standard |
| sample | [fmol] | [fmol] |
| 1 | — | 1000 |
| 2 | 2 | 1000 |
| 3 | 10 | 1000 |
| 4 | 50 | 1000 |
| 5 | 100 | 1000 |
| 6 | 1000 | 1000 |
| 7 | 5000 | 1000 |
| 8 | 20000 | 1000 |

The calibration samples then undergo tryptic digestion, and are elsehow treated like the “real” test samples, e.g., if applicable, reaction can be halted by addition of an acid such as TFA, sample can be purified by solid phase extraction, and can be lyophilized and resuspended for LC-MS/MS analysis.

According to one embodiment of the method according to the invention, a calibration curve is generated based on the ratio of the spectrometry signals of the peptides derived from digestion of the MHC molecule standard (also called “MRF-derived peptides)” vs. the peptides from the internal standard are then calculated and plotted.

The ratio of the MS signals of the MRF-derived peptides vs. the peptides from the internal standard are then calculated and plotted. For this purpose, the total amount of MRF per digestion is plotted on the x-axis versus the ratio of the unlabelled monomer-derived peptide MS area divided by the area of the corresponding isotopically labelled internal standard (see FIG. 7B). Each MRF peptide quantity translates into a certain MS ratio compared to the IS added to the sample at constant concentrations.

Generally, the quantities of MHC can be directly inferred from their peptide levels, due to the 1:1 stoichiometry between a given peptide in the sample obtained by digestion and the MHC protein that was in the sample prior to digestion.

According to one embodiment of the method according to the invention, the MHC concentration is calculated based on the normalized protein concentration (1/μg).

Transformation of each peptide-specific calibration curve equation allows to calculate the peptide concentration of a given analyte peptide, in case that the internal standard was spiked in at the same concentration as for the calibration curve:

$$Peptide concentration [ f mol / μ g ] = \frac{( \frac{MS ratio - b}{a} )}{Digested protein amount [ e . g \text{.20} μg ]} \qquad Equation 1$$

in which “a” and “b” are as shown in FIG. 7.

In such way, the concentration of each MHC peptide can be derived and expressed, e.g., in fmol/μg.

According to one embodiment of the method according to the invention, the concentration of the MHC protein vs. the test sample volume is calculated based on the total protein concentration in the test sample prior to digestion.

In such way, the concentration of each MHC peptide can be transformed into fmol/μL, taking the total protein concentration per lysate into account if the latter has been determined previously, e.g. via BCA assay:

$$Peptide concentration [ \frac{f mol}{μ g} ] * Total protein concentration [ \frac{μ g}{μ L} ] = Peptide concentration [ \frac{f mol}{μ L} ] \qquad Equation 2$$

According to one embodiment of the method according to the invention, the number of MHC molecules per cell in the test sample is calculated based on the total cell count in the sample.

To further translate the peptide concentration from fmol/μL into total protein copies per lysate, the overall lysate volume and the cell count per lysate along with Avogadro's constant have to be further taken into account:

$$\frac{Copies}{cell} = \frac{\begin{matrix}Peptide concentration [ \frac{f mol}{μ g} ] * \\ Total lysate volume [ μ L ] * \\ 6 , 022 \frac{{10}^{23}}{{10}^{15} [ f mol ]}\end{matrix}}{cell count} \qquad Equation 3$$

According to another aspect of the invention, a set of three or more peptides—also called “sample peptide analogues”—is provided, wherein the sequence of each peptide corresponds to a stretch, domain, or epitope of one HLA allotype selected from the group consisting of HLA-A, HLA-B, HLA-C, and/or HLA-E. This set of three or more peptides makes up the internal standard that is discussed elsewhere herein.

With regard to these subtypes, see the further discussion elsewhere herein in connection with the method of the invention. Advantages and characteristics will not be repeated here to avoid lengthiness This and the following sets of peptides are used for the internal standard (IS) as described hereinabove.

According to one embodiment of the peptide set according to the invention, the sequence of each peptide corresponds to a stretch, domain, or epitope of one HLA allotype selected from the group consisting of HLA-A, HLA-B, HLA-C, and/or HLA-E.

According to one embodiment of the peptide set according to the invention, the HLA to a stretch, domain, or epitope of which the sequences of the peptides correspond is HLA-A*02.

According to one embodiment of the peptide set according to the invention, the HLA to a stretch, domain, or epitope of which the sequences of the peptides correspond is at least one selected from the group consisting of HLA-A*02:01; HLA-A*01:01; HLA-A*03:01; HLA-A*24:02; HLA-B*07:02; HLA-B*08:01; HLA-B*44:02 and/or HLA-B*44:03.

In one embodiment, these peptides are selected from the group consisting of
SEQ ID NO 1-10 (or their counterparts comprising overhangs, SEQ ID NO 18-27), and
SEQ ID NO 44-62 (or their counterparts comprising overhangs, SEQ ID NO 63-81)

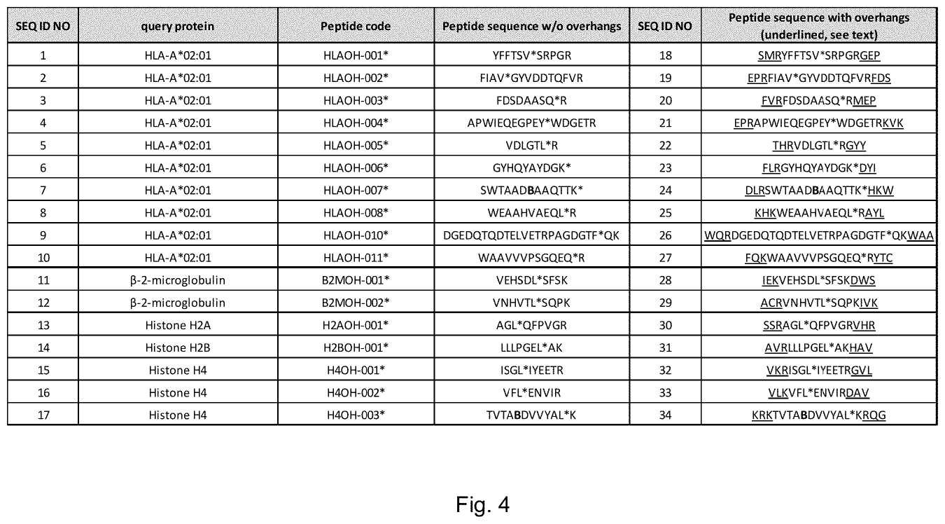

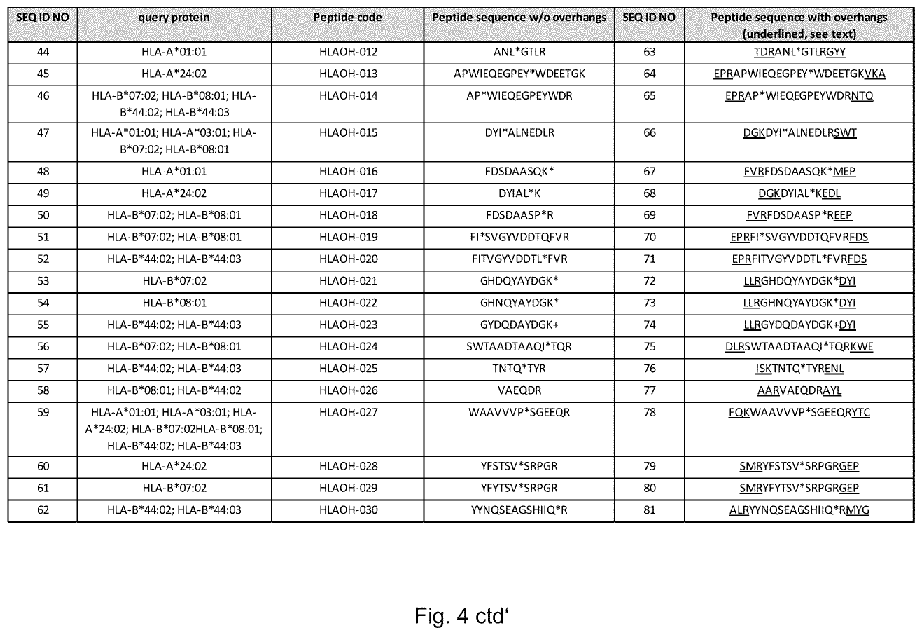

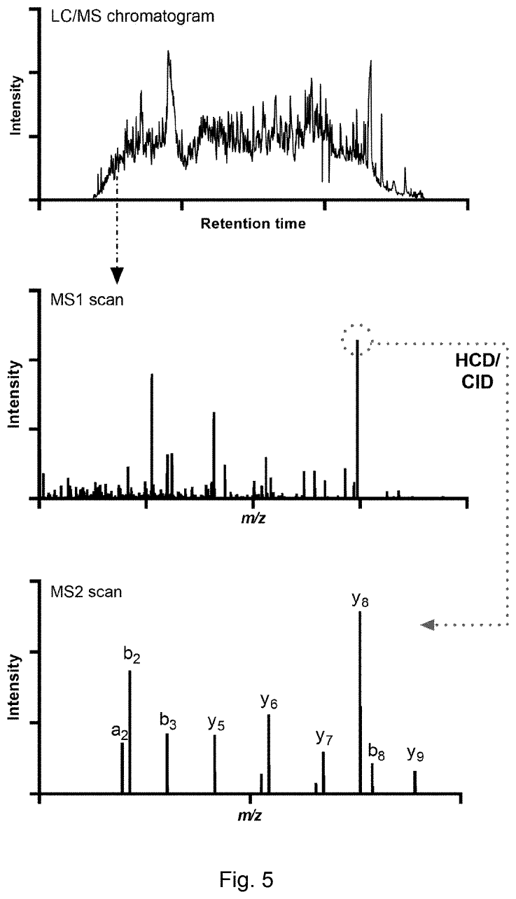

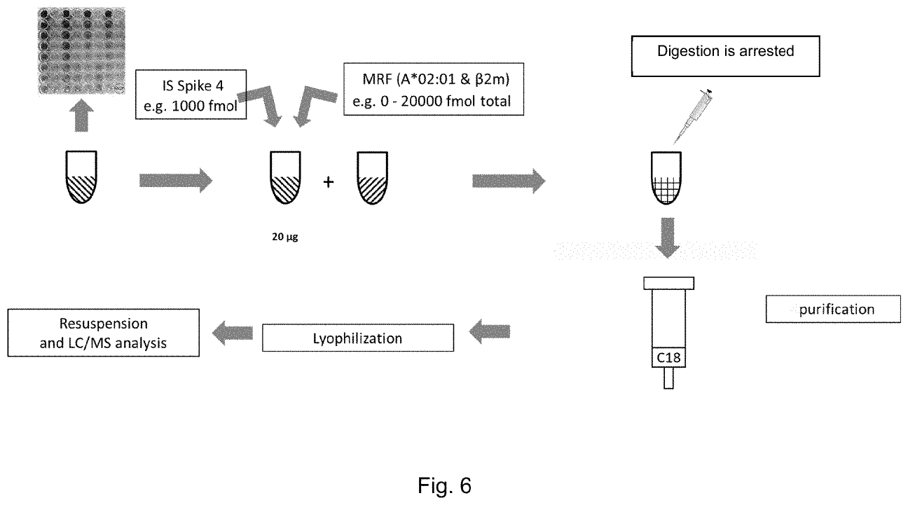

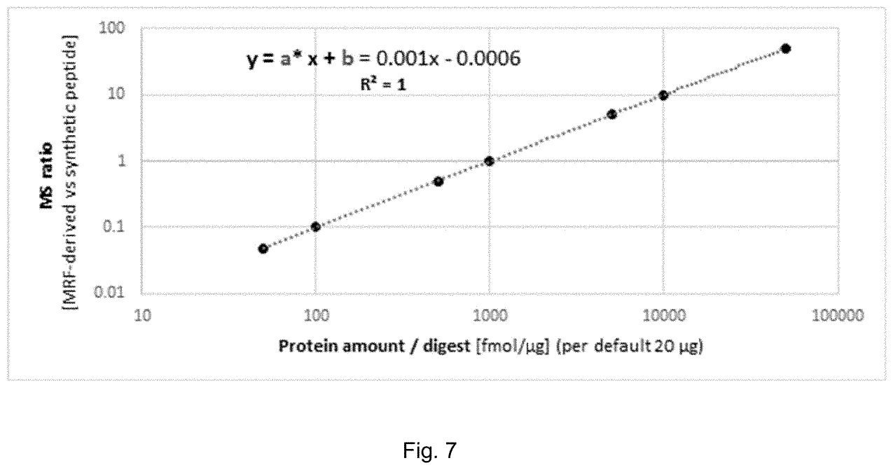

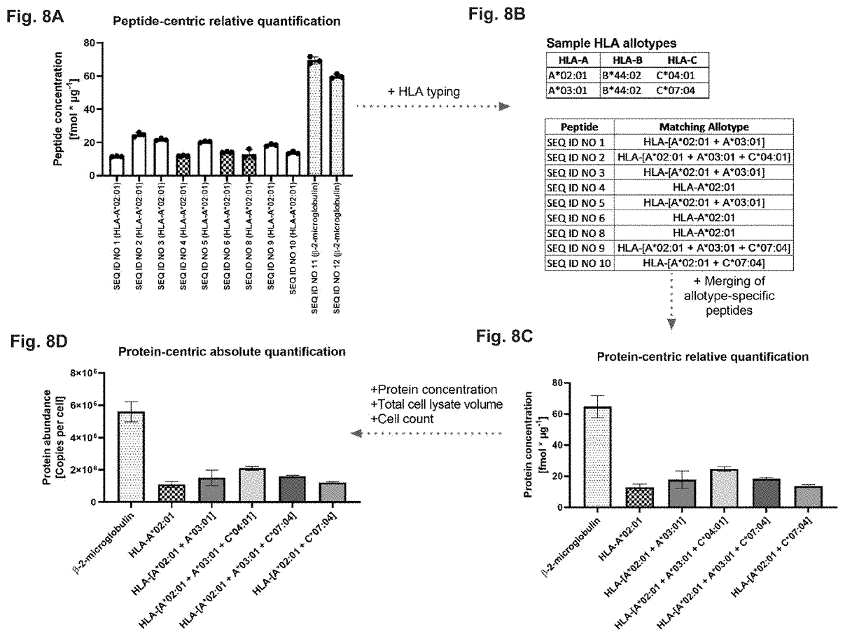

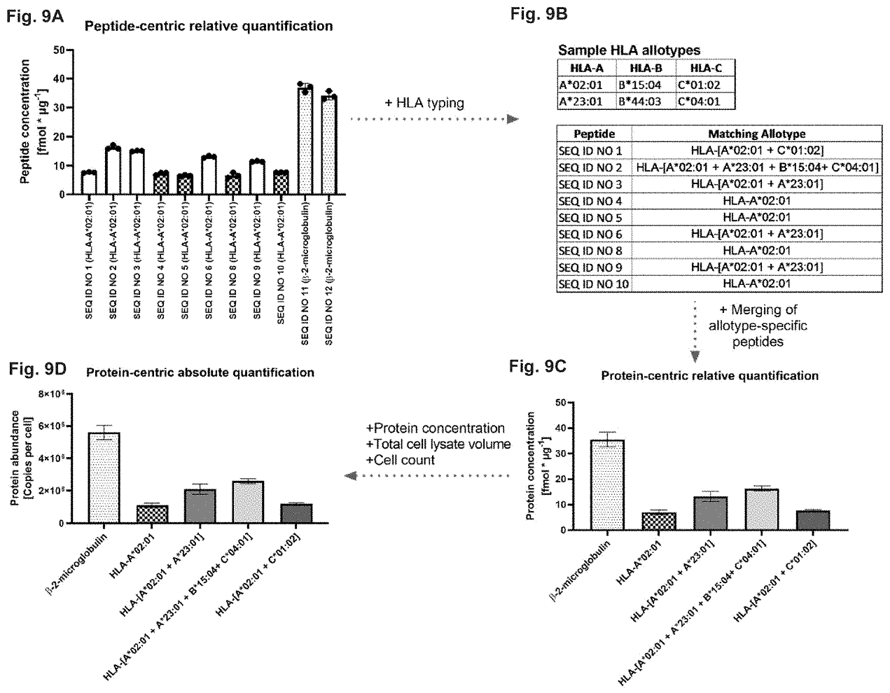

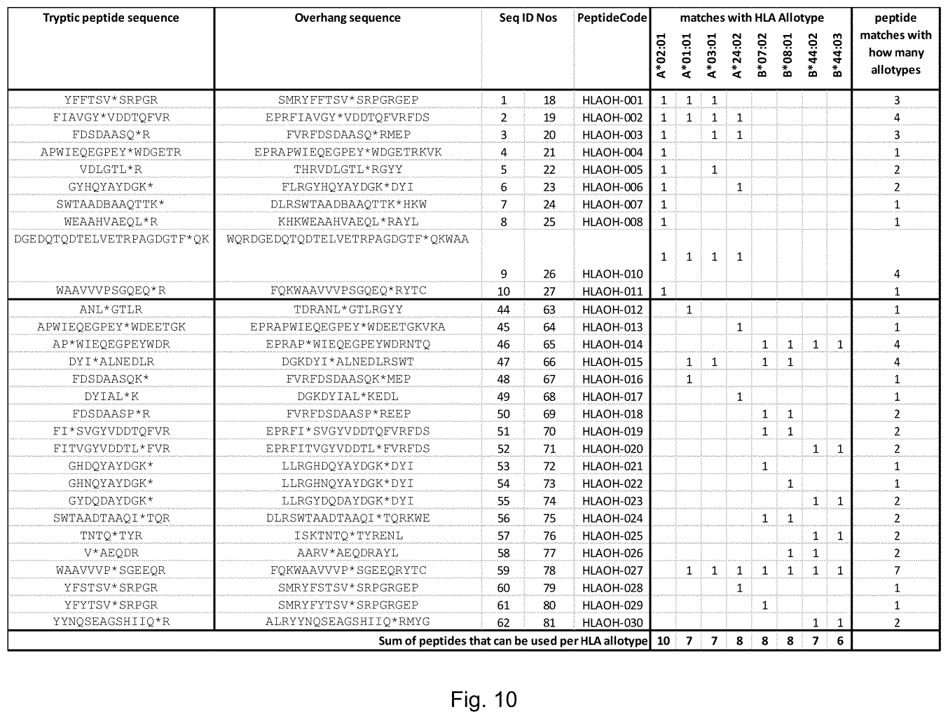

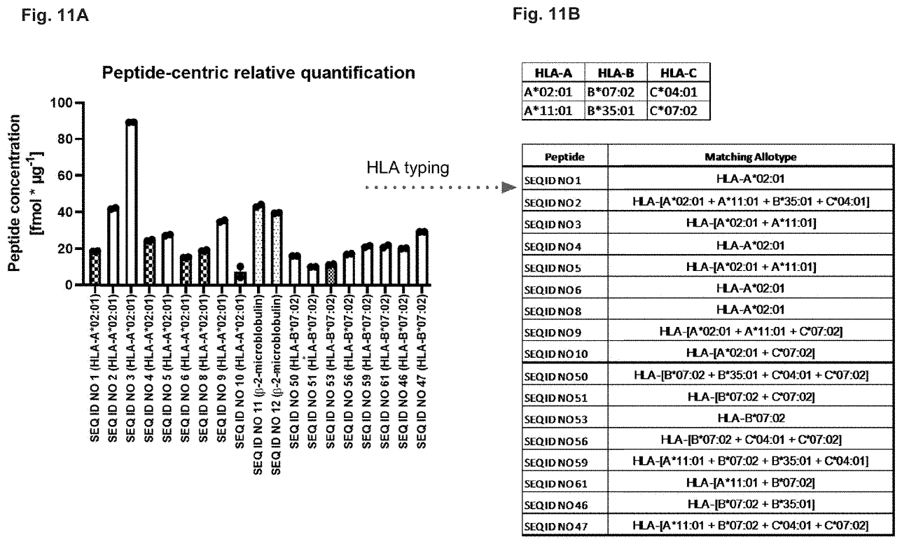

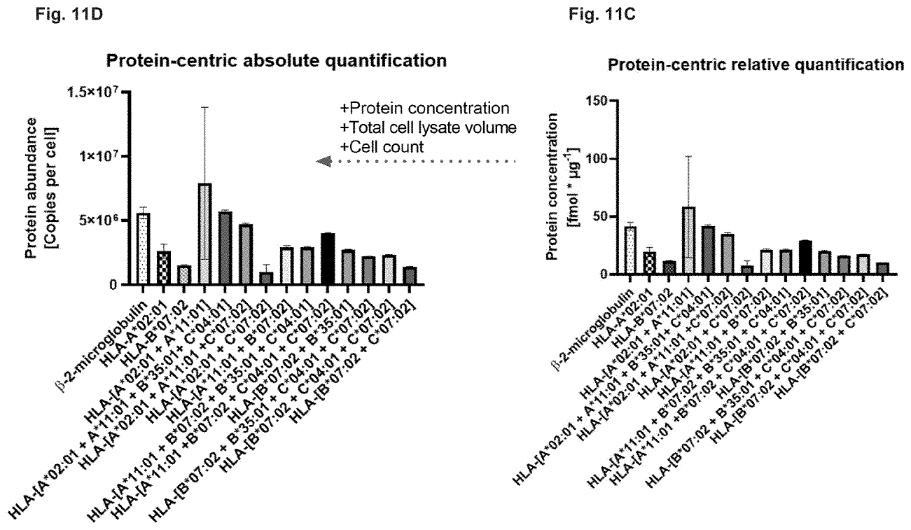

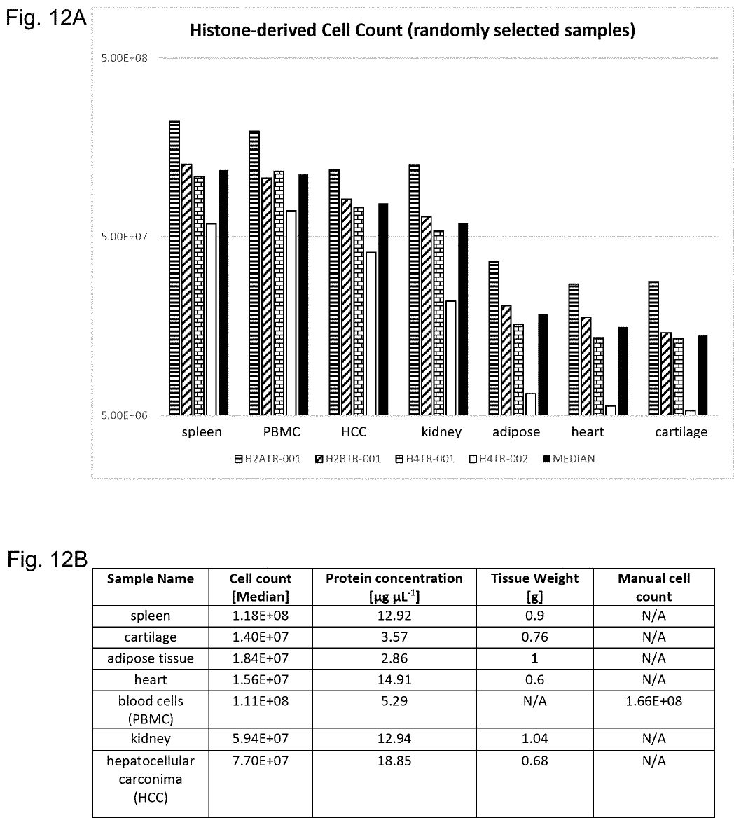

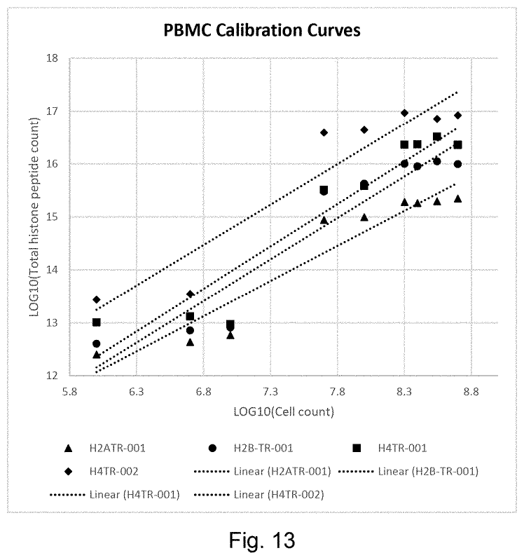

See also FIG. 4 and FIG. 10 and also Table 1 and 4 for further information regarding the specificity and match of these peptides. Generally, preferred peptide sets that can be used in the context of the present invention are e.g. disclosed in FIGS. 8, 9 and 10.

According to one embodiment of the peptide set according to the invention, the HLA to a stretch, domain, or epitope of which the sequences of the peptides correspond is HLA-A*02:01.

In one embodiment, these peptides are selected from the group consisting of SEQ ID NO 1-10 (or their counterparts comprising overhangs, SEQ ID NO 18-27). See also FIG. 4 and FIG. 10 and also Table 1 or further information regarding the specificity and match of these peptides.

With regard to this subtype, see the further discussion elsewhere herein in connection with the method of the invention. Advantages and will not be repeated here to avoid lengthiness.

In the following, some embodiments of the set of peptides are described. Advantages and characteristics of these embodiments are already discussed hereinabove in the context of the method of the invention and will not be repeated here to avoid lengthiness.

According to one embodiment, the set comprises at least two peptides having a sequence which corresponds to a stretch, domain, or epitope of at least two different HLA allotypes. In this embodiment, the method enables quantification of a further HLA allotype. According to one embodiment, a further set of three or more peptides is used whose sequence corresponds to a stretch, domain, or epitope of such further HLA allotype.

Optionally, the set can comprise one or more further sets of between ≥5 and ≤20 further peptides the sequence of which corresponds to a stretch, domain, or epitope of another HLA allotype different to HLA-A*02:01. In such way, more than one HLA allotype can be quantified.

According to one embodiment, the sequence of at least one of the peptides of the set has been derived from the template protein by in silico protease digestion.

According to one embodiment, at least one peptide of the set is selected in such way that it does not comprise C (Cys) residues. According to one embodiment, at least one peptide of the set is selected in such way that it does not comprise M (Met) residues. According to one embodiment, at least one peptide of the set is selected in such way that it does not comprise post-translational modifications, such as N-glycosylation. According to one embodiment, the peptides of the set are produced synthetically.

According to one embodiment, the peptides of the set have a length, not including potential overhangs, of between ≥4 and ≤50 AA. According to one embodiment, the peptides of set have a molecular weight, not including potential overhangs, of between ≥500 and ≤4000 Da.

According to one embodiment, at least one peptide in the set is labelled. According to one embodiment, the label is at least one of a metal-coded tag and/or an isotope label.

In one example, the set comprising peptides the sequence of which corresponds to a stretch, domain, or epitope of the following proteins, as shown in the following table:

| | | Number of |
|---|---|---|
| | | different |
| Template protein | Example | peptides in IS |
| beta-2-micro- | | ≥1-≤4 |
| globulin | | |
| HLA | HLA-A*02:01 | ≥2-≤20 |
| protein which is | Histone, e.g., | ≥2-≤10 |
| roughly | at least one | |
| proportional | of Histone H2A, | |
| to the total | Histone H2B, | |
| number of cells | and Histone H4 | |

Optionally, the set can comprise one or more further sets of ≥3-≤20 further peptides the sequence of which corresponds to a stretch, domain, or epitope of another HLA allotype different to HLA-A*02:01. In such way, more than one HLA allotype can be quantified.

Sets of peptides for internal standard to quantify HLA-A*02:01

According to one embodiment, the set comprises at least one of:
5, 6, 7, 8, 9, or 10 peptides each of which comprising an amino acid selected from the group consisting of any one of SEQ ID NO: 1-SEQ ID NO: 10, and/or
5, 6, 7, 8, 9, or 10 peptides each of which comprising an amino acid selected from the group consisting of any one of SEQ ID NO: 18-SEQ ID NO: 27.

Note that, in all sets of sample peptide analogues, peptides with overhangs can be replaced by the non-overhang counterparts and vice versa. E.g., instead of the peptide of SEQ ID NO 1, also the peptide of SEQ ID NO 18 can be used, or instead of the peptide of SEQ ID NO 27, also the peptide of SEQ ID NO 10 can be used.

It should also be self explaining that, instead of using the respective peptides with overhangs (SEQ ID NOs 18 and 27), peptides with even longer N- and C terminal overhangs can be used, as long as these peptides yield the same peptides after protease digestion.

According to one embodiment of the peptide set according to the invention, the set further comprises at least one peptide the sequence of which corresponds to a stretch, domain, or epitope of beta-2-microglobulin (β2m).

In one embodiment, these peptides are selected from the group consisting of SEQ ID NO 11-12 (or their counterparts comprising overhangs, SEQ ID NO 28-29). See also FIG. 4 and also Table 2 or further information regarding the specificity and match of these peptides.

With regard to this embodiment, see the further discussion elsewhere herein in connection with the method of the invention. Advantages and will not be repeated here to avoid lengthiness.

According to one embodiment, the set comprises at least one of:
1 or 2 peptides each of which comprising an amino acid selected from the group consisting of any one of SEQ ID NO: 11-SEQ ID NO: 12, and/or
1 or 2 peptides each of which comprising an amino acid selected from the group consisting of any one of SEQ ID NO: 28-SEQ ID NO: 29.

Note that, in all sets of sample peptide analogues, peptides with overhangs can be replaced by the non-overhang counterparts and vice versa. E.g., instead of the peptide of SEQ ID NO 11, also the peptide of SEQ ID NO 28 can be used, or instead of the peptide of SEQ ID NO 29, also the peptide of SEQ ID NO 12 can be used.

It should also be self-explaining that, instead of using the respective peptides with overhangs (SEQ ID NOs 28 and 29), peptides with even longer N- and C terminal overhangs can be used, as long as these peptides yield the same peptides after protease digestion.

According to one embodiment of the peptide set according to the invention, the set further comprises at least one peptide the sequence of which corresponds to a stretch, domain, or epitope of one or more proteins the abundance of which is roughly proportional to the total number of cells in the sample.

With regard to the term a “protein the abundance of which is roughly proportional to the total number of cells in the sample” see the further discussion elsewhere herein in connection with the method of the invention. Advantages and characteristics will not be repeated here to avoid lengthiness.

According to one embodiment of the peptide set according to the invention, at least one protein the abundance of which is roughly proportional to the total number of cells in the sample is a histone, e.g., H2A, H2B or H4.

In one embodiment, these peptides are selected from the group consisting of SEQ ID NO 13-17 (or their counterparts comprising overhangs, SEQ ID NO 30-34). See also FIG. 4 and also Table 3 or further information regarding the specificity and match of these peptides.

Advantages and characteristics of these embodiments are already discussed hereinabove in the context of the method of the invention and will not be repeated here to avoid lengthiness.

Hence, quantification of a protein the abundance of which is roughly proportional to the total number of cells in the sample can be used to quantify the total amount of cells in the sample, and hence, assess the mean abundance of HLA per cell.

According to one embodiment of the peptide set according to the invention, the sequence of at least one peptide in the set comprises an overhang of amino acids at least the N-terminus and/or at the C-terminus, wherein the overhang of amino acids comprises a protease cleavage site.

Said protease cleavage site is, in one embodiment, a trypsin cleavage site, as disclosed elsewhere herein.

As used herein, the term “overhang of amino acids” means that the peptides are selected in such way that comprise one or more further amino acid residues beyond at least the C- or N-terminal cleavage site of the protease that has been used for the template protein digestion.

Advantages and characteristics of this embodiment are already discussed hereinabove in the context of the method of the invention and will not be repeated here to avoid lengthiness.

According to one embodiment, the set comprises at least one peptide comprising an amino acid sequence selected from the group consisting of SEQ ID No 1-SEQ ID NO 34 and SEQ ID No 44-SEQ ID NO 81. It may furthermore comprise 2, 3, 4, 5, 6, 7, 8, 9, 10, 11, 12, 13, 14, 15, 16, 17, 18, 19, 20, 21, 22, 23, 24, 25, 26, 27, 28, 29, or 30 peptides comprising an amino acid sequence selected from the group consisting of SEQ ID No 1-SEQ ID NO 34 and SEQ ID No 44-SEQ ID NO 81.

It should also be self-explaining that, instead of using the respective peptides with overhangs (SEQ ID NOs 18-34 and 63-81), peptides with even longer N- and C terminal overhangs can be used, as long as these peptides yield the same peptides after protease digestion (i.e., the peptides of SEQ ID NOs 1-17 and 44-62).

Based on these peptides, and the information disclosed in FIGS. 4 and 10, and also in Tables 1 and 4, the skilled person can assemble sets of sample peptide analogues for the quantification of different HLA allotypes in a sample either individually, or simultaneously.

In order to allow absolute quantification, the sample peptide analogues of FIG. 4 that are derived from ß2 microglobulin and/or the histones (see also tables 2 and 3) can be added to the set of sample peptide analogues.

Hence, FIGS. 4 and 10, together with tables 1-4, provide a toolbox that allows the relative of absolute quantification of one or more HLA allotypes in a given sample.

Sets of sample peptide analogues in internal standard for quantifying HLA-A*02:01

According to one embodiment, the set comprises at least one of:
5, 6, 7, 8, 9, or 10 peptides each of which comprising an amino acid selected from the group consisting of any one of SEQ ID NO: 1-SEQ ID NO: 10, and/or
5, 6, 7, 8, 9, or 10 peptides each of which comprising an amino acid selected from the group consisting of any one of SEQ ID NO: 18-SEQ ID NO: 27.

According to one embodiment, the set comprises at least one of:
1 or 2 peptides each of which comprising an amino acid selected from the group consisting of any one of SEQ ID NO: 11-SEQ ID NO: 12, and/or
1 or 2 peptides each of which comprising an amino acid selected from the group consisting of any one of SEQ ID NO: 28-SEQ ID NO: 29.

According to one embodiment, the set comprises at least one of:
1, 2, 3, 4, or 5 peptides each of which comprising an amino acid selected from the group consisting of any one of SEQ ID NO: 13-SEQ ID NO: 17, and/or
1, 2, 3, 4, or 5 peptides each of which comprising an amino acid selected from the group consisting of any one of SEQ ID NO: 30-SEQ ID NO: 34.

Note that, in all sets of sample peptide analogues, peptides with overhangs can be replaced by the non-overhang counterparts and vice versa. E.g., instead of the peptide of SEQ ID NO 13, also the peptide of SEQ ID NO 30 can be used, or instead of the peptide of SEQ ID NO 33, also the peptide of SEQ ID NO 16 can be used.

According to one embodiment, the set comprises:
5, 6, 7, 8, 9, or 10 peptides each of which comprising an amino acid selected from the group consisting of any one of SEQ ID NO: 1-SEQ ID NO: 10 and SEQ ID NO: 18-SEQ ID NO: 27,
1 or 2 peptides each of which comprising an amino acid selected from the group consisting of any one of SEQ ID NO: 11-SEQ ID NO: 12, and SEQ ID NO: 28-SEQ ID NO: 29
1, 2, 3, 4, or 5 peptides each of which comprising an amino acid selected from the group consisting of any one of SEQ ID NO: 13-SEQ ID NO: 17 and SEQ ID NO: 30-SEQ ID NO: 34.

Note that, in all sets of sample peptide analogues, peptides with overhangs can be replaced by the non-overhang counterparts and vice versa. E.g., instead of the peptide of SEQ ID NO 11, also the peptide of SEQ ID NO 28 can be used, or instead of the peptide of SEQ ID NO 29, also the peptide of SEQ ID NO 12 can be used.

According to one embodiment, at least one of the peptides consists of the respective sequence.

This means, in the context of the present invention, that the respective peptide has the exact same length as the respective sequence. According to one embodiment, 2, 3, 4, 5, 6, 7, 8, 9, 10, 11, 12, 13, 14, 15, 16, or 17 of the peptides consist of the respective sequence.

According to one embodiment all of the peptides consist of the respective sequences.

Sets of sample peptide analogues in internal standard for quantifying HLA-A*02:01 and/or other HLA allotypes

According to one embodiment, the set comprises at least one of:
5, 6, 7, 8, 9, or 10 peptides each of which comprising an amino acid selected from the group consisting of any one of SEQ ID NO: 1-SEQ ID NO: 10 and SEQ ID NO: 44-SEQ ID NO: 62, and/or
5, 6, 7, 8, 9, or 10 peptides each of which comprising an amino acid selected from the group consisting of any one of SEQ ID NO: 18-SEQ ID NO: 27 and SEQ ID NO: 63-SEQ ID NO: 81, and/or.

According to one embodiment, the set comprises at least one of:
1 or 2 peptides each of which comprising an amino acid selected from the group consisting of any one of SEQ ID NO: 11-SEQ ID NO: 12, and/or
1 or 2 peptides each of which comprising an amino acid selected from the group consisting of any one of SEQ ID NO: 28-SEQ ID NO: 29.

According to one embodiment, the set comprises at least one of:
1, 2, 3, 4, or 5 peptides each of which comprising an amino acid selected from the group consisting of any one of SEQ ID NO: 13-SEQ ID NO: 17, and/or
1, 2, 3, 4, or 5 peptides each of which comprising an amino acid selected from the group consisting of any one of SEQ ID NO: 30-SEQ ID NO: 34.

Note that, in all sets of sample peptide analogues, peptides with overhangs can be replaced by the non-overhang counterparts and vice versa. E.g., instead of the peptide of SEQ ID NO 13, also the peptide of SEQ ID NO 30 can be used, or instead of the peptide of SEQ ID NO 33, also the peptide of SEQ ID NO 16 can be used.

According to one embodiment, the set comprises:
5, 6, 7, 8, 9, or 10 peptides each of which comprising an amino acid selected from the group consisting of any one of SEQ ID NO: 1-SEQ ID NO: 10 and SEQ ID NO: 44-SEQ ID NO: 62, and/or
5, 6, 7, 8, 9, or 10 peptides each of which comprising an amino acid selected from the group consisting of any one of SEQ ID NO: 18-SEQ ID NO: 27 and SEQ ID NO: 63-SEQ ID NO: 81, and/
1 or 2 peptides each of which comprising an amino acid selected from the group consisting of any one of SEQ ID NO: 11-SEQ ID NO: 12, and SEQ ID NO: 28-SEQ ID NO: 29
1, 2, 3, 4, or 5 peptides each of which comprising an amino acid selected from the group consisting of any one of SEQ ID NO: 13-SEQ ID NO: 17 and SEQ ID NO: 30-SEQ ID NO: 34.

In the following, a second aspect of the present invention will be discussed, which relates to a novel and inventive method of determining cell count in a sample. Such method can for example be used to determine the amount of cells to be attacked in a diagnosed tumor, and thus helps to determine a personalized therapeutic window. It may also help to determine the total number or treatable targets in a given tissue, when the target density per cell is known.

Technology-wise, this method has large overlaps with the method of the first aspect as discussed above, according to which the MHC content in a sample is quantified. Therefore, preferred embodiments discussed in the context of the second aspect of the invention are deemed to be also disclosed with regard to the first. aspect, and vice versa.

According to this second aspect a method of determining the cell count in a test sample comprising at least one cell, is provided. The method comprises at least the steps of:
a) homogenizing the sample,
b) digesting the homogenized sample with a protease, before or after addition of the internal standard
c) subjecting the digested sample to a step of chromatography and/or spectrometry analysis, and
d) determining the content of at least one histone in the digested sample, and
e) determining, on the basis thereof, the cell count in the sample.

The sample is preferably a sample taken from a subject preferably from a human subject. The sample may for example have been taken by a biopsy, or may be a liquid sample (urine, blood, semen, liquor, lymph fluid).

In different embodiments the sample is a sample taken from a healthy tissue, or is a sample taken from a neoplastic tissue or liquid sample. e.g., Sarcoma, Carcinoma, Lymphoma, and Leukaemia.

According to one embodiment, the sample is purified after step b) and prior to step c).

According to one embodiment, the histone is at least one selected from the group consisting of histone H2A, histone H2B, or histone H4.

According to one embodiment, the content of at least two histones is determined, wherein the two histones are selected from group consisting of histone H2A, histone H2B, or histone H4.

According to one embodiment, the content of three histones is determined, wherein the histones are histone H2A, histone H2B, and histone H4.

According to one embodiment, the method further comprises adding an internal standard to the sample.

According to one embodiment, the internal standard comprises at least one peptide in a defined concentration.

According to one embodiment, the sequence of the at least one peptide corresponds to a stretch, domain, or epitope of one histone selected from the group consisting of histone H2A, histone H2B, or histone H4.

According to one embodiment, the internal standard comprises at least two peptides in defined concentrations. Preferably, the sequences of each of the two or more peptides correspond to a stretch, domain, or epitope of two or more respective histones selected from the group consisting of histone H2A, histone H2B, or histone H4.

According to one embodiment, the internal standard comprises at least three peptides in defined concentrations. Preferably, the sequences of each of the three or more peptides correspond to a stretch, domain, or epitope of three respective histones selected from the group consisting of histone H2A, histone H2B, or histone H4.

According to one embodiment, the at least one peptide in the internal standard comprises an amino acid sequences selected from the group consisting of SEQ ID NO 13, SEQ ID NO 14, SEQ ID NO 15, SEQ ID NO 16, SEQ ID NO 17, SEQ ID NO 30, SEQ ID NO 31, SEQ ID NO 32, SEQ ID NO 33 and/or SEQ ID NO 34.

In this context, it is important to mention that SEQ ID NO 13, SEQ ID NO 14, SEQ ID NO 15, SEQ ID NO 16, SEQ ID NO 17 relate to peptides that are eventually determined in the sample after which the latter has been digested by use of the protease. Instead of these peptides, peptides can be used which comprises N- and C terminal overhangs that are actually removed by the protease digestion. SEQ ID NO 30, SEQ ID NO 31, SEQ ID NO 32, SEQ ID NO 33 and SEQ ID NO 34 represent such peptides which, when subjected to trypsin treatment, are cleaved so as to yield the peptides of SEQ ID NO 13, SEQ ID NO 14, SEQ ID NO 15, SEQ ID NO 16, SEQ ID NO 17.

It should be self-explaining that, instead of using the peptides of SEQ ID NO 30, SEQ ID NO 31, SEQ ID NO 32, SEQ ID NO 33 and SEQ ID NO 34, peptides with even longer N- and C terminal overhangs can be used, as long as these peptides yield the same peptides of SEQ ID NO 13, SEQ ID NO 14, SEQ ID NO 15, SEQ ID NO 16, SEQ ID NO 17 after protease digestion.

Preferred peptide sets that can be used in the context of the invention are e.g. shown in FIG. 11.

According to one embodiment, at least one peptide of the internal standard is selected in such way that it does not comprise C residues.

C (Cys) comprises a thiol group which has the potential to build disulphide bridges with other cysteines in the same or other peptides. Hence, having cysteine comprising peptides in the internal standard could lead to artifacts caused by the formation of heterooligomers, and hence errors in the analysis.

According to one embodiment, at least one peptide of the internal standard is selected in such way that it does not comprise M residues. M (Met) comprises a thioether, and partly oxidizes during sample preparation, which hence leads to the generation of two different peptides (reduced M and oxidized M oxidized), both of which would have to be quantified.

As an alternative, M is replaced by methionine sulfoxide (MetO), for which the one letter code “B” is used herein.

According to one embodiment, at least one peptide of the internal standard is selected in such way that it does not comprise post-translational modifications.

This applies, inter alia, to as N-glycosylation. N-glycosylation motifs are NXS and NXT, so in this embodiment, care is taken that the peptides used for the internal standard do not comprise any of these motifs.

Other post-translational modifications that can preferably be avoided by respective selection of the peptides used for the internal standard (and avoidance of amino acid residues that are likely subject of such post-translational modifications) include, but are not limited to
mono, di- or trimethylation of e.g., lysine or arginine,
acetylation of e.g. lysine or asparagine, or
phosphorylation of e.g. tyrosine, threonine or serine.

According to one embodiment, prior or after homogenization, the sample is not treated with, or obtained by, immunoprecipitation.

According to one embodiment, prior the protease used for digesting the sample is trypsin.

According to one embodiment, the test sample is selected from the group consisting of
an extract of a biological sample comprising proteins
a primary, non-cultured sample, and/or
sample obtained from one or more cell lines.

According to one embodiment, the step of chromatography and/or spectrometry analysis comprises LC-MS/MS analysis.

According to one embodiment, the method further comprises the provision of a calibration table, calibration curve or calibration algorithm which has been established by
a) providing at least two samples of suspended, dispersed or otherwise countable cells, in which at least two samples the concentration of cell is different
b) determining the cell count in said at least two samples,
c) determining the content of at least one histone in the at least two samples according to the method of any one of claims 41-49, and
d) establishing a calibration table, calibration curve or calibration algorithm by correlating, in the at least two samples, the histone content with the cell count.

Such method can, for example, titration of one or more cells vs. a histone-based signal, as obtained by the spectrometry methods disclosed herein. More precisely, the ratio of endogenous histone peptides obtained by tryptic digestion versus their heavy isotope-labelled internal standard peptides is determined, and the resulting histone content is the correlated with the cell count.

According to several embodiments, the cell count in said sample is determined by at least one method selected from the group of:

manual (optical) counting automated counting by means of a cell counter counting by means of image analysis

Generally, there are several methods for cell counting. Manual (optical) counting is oftentimes performed using a counting chamber, which is a microscope slide that is especially designed to enable cell counting. Hemocytometers and Sedgewick Rafter counting chambers are two types of counting chambers. The hemocytometer has two gridded chambers in its middle, which are covered with a special glass slide when counting. A drop of cell culture is placed in the space between the chamber and the glass cover, filling it via capillary action. Looking at the sample under the microscope, the researcher uses the grid to manually count the number of cells in a certain area of known size. The separating distance between the chamber and the cover is predefined, thus the volume of the counted culture can be calculated and with it the concentration of cells. Cell viability can also be determined if viability dyes are added to the fluid.

For automated cell counting, a coulter counter is oftentimes used. This an appliance that can count cells as well as measure their volume. It is based on the fact that cells show great electrical resistance; in other words, they conduct almost no electricity. In a Coulter counter the cells, swimming in a solution that conducts electricity, are sucked one by one into a tiny gap. Flanking the gap are two electrodes that conduct electricity. When no cell is in the gap, electricity flows unabated, but when a cell is sucked into the gap the current is resisted. The Coulter counter counts the number of such events and also measures the current (and hence the resistance), which directly correlates to the volume of the cell trapped. A similar system is the CASY cell counting technology. As an alternative, flow cytometry can be used. Therein, the cells flow in a narrow stream in front of a laser beam. The beam hits them one by one, and a light detector picks up the light that is reflected from the cells.

For counting by means of image analysis, high-quality microscopy images are used which are then analysed by a digital image processer, which for example detects cell borders and/or nuclei, and ten applies statistical classification algorithms to perform automated cell detection and counting as an image analysis task.

According to several embodiments, the cells in the sample of suspended, dispersed or otherwise countable cells are at least one of
diploid cells, and/or
mononuclear cells.

In such way, it is ensured that the histone content that is determined is representative for a typical cell type.

According to one embodiment, the sample of suspended, dispersed or otherwise countable cells is a blood sample.

Preferably, the blood sample comprises, or essentially consists of, PBMC (Peripheral Blood Mononuclear Cells).

In other embodiments, the cells in the sample of suspended, dispersed or otherwise countable cells an be other cells types that have been isolated and brought into suspension, e.g., by means of enzymatic digestion of the extracellular matrix. Such cells comprise, inter alia, suspended hepatocytes, suspended ovary cells and the like.

It is in this context important to mention that the method according to the present invention differs substantially from a method disclosed in Edfors et al. (2016). Therein, the authors do not consider any protein/histone losses or incomplete cell lysis during sample processing. The absolute amount of histones (as determined via spike-in of an internal standard) is translated into the number of cells via the integration of the “number of histones per cell” (see Error! Reference source not found.; equation 5). This total histone count value is an arbitrary value since it assumes a 1:1 correlation of DNA with histones, thus the state that all histones are bound to DNA and for example no unbound histones are taken into account (Edfors et al., 2016).

Contrary thereto, the method according to the invention takes such these losses during processing into account.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus.

### Example 1

As a biological source of MHC proteins in cell lines, human acute myeloid leukemia cell line MUTZ-3 was used.

A total of 500×10⁶ cells were collected and subjected to cell lysis in a CHAPS detergent-containing buffer and homogenized assisted by sonification. Insoluble compounds were removed by ultracentrifugation and the cleared lysate was stored at −80° C. until further processing.

Prior to further downstream analysis, the protein concentration in the cleared lysate was determined using BCA assay. A titration series of bovine serum albumin in 50 mM ammonium bicarbonate was used as a calibration curve to calculate total protein concentration in the cell lysate. The protein concentration in the cleared lysate was found to be at 13.4 μg

Prior to sample digestion, the internal standard mix containing the relevant overhang peptides as shown in Table 1 (optionally also Tables 2-4) at a stock concentration of 25 pmol μ⁻¹ was diluted to 100 fmol μ⁻¹ using 50 mM ammonium bicarbonate as a diluent.

Subsequently, proteolytic digestion was initiated by adding 150 μL SMART Digest buffer, 10 μL 100 fmol μ⁻¹ internal standard mix, 20 μg total protein from MUTZ-3 cell line lysate (i.e. 1.49 μL lysate at 13.4 μg μ⁻¹ as determined previously) to the corresponding SMART Digest trypsin aliquot vial. Finally, H₂O_{dd} was added to a final total volume of 200 μL and the reaction tube was stirred for 3 sec.

The sample was transferred to a pre-heated heating block and efficient proteolytic digestion initiated by incubation at 70° C. for 90 min at 1,400 rpm. In order to denature trypsin afterwards and thus irreversibly stop the proteolytic digestion, TFA was added to the reaction tube at a final concentration of 0.5%, which lowered the pH to <3.

For sample clean-up (i.e. removal of salts and remaining high-molecular weight compounds such as trypsin beads) prior to LC-MS/MS analysis, C18 reverse-phase solid-phase extraction was used employing 0.1% TFA as a wash solvent of the C18-bound peptides. After peptide elution using 70% ACN, the sample was lyophilized to complete dryness and subsequently reconstituted in 5% FA at a concentration of 500 ng μL⁻¹.

The peptide mixture was then subjected to LC-MS/MS using a nanoACQUITY UPLC system (Waters) coupled online to an Orbitrap Fusion™ Tribrid™ mass spectrometer (Thermo Fisher Scientific) at a flow rate of 300 n1 min⁻¹. Data were acquired in three technical replicates and a total of 250 ng sample was loaded onto the column per LC-MS/MS run.

The mass spectrometer was operated in scheduled parallel reaction monitoring (sPRM) mode to allow for the targeted analysis of the pre-selected probe set. Nano-flow sPRM assays were performed using a 42 min three-step linear, binary gradient consisting of solvent A (0.1% FA in H₂O) and solvent B (0.1% FA in ACN).

For successful peptide ion dissociation, higher-energy collisional dissociation (HCD) was employed at a normalized collision energy (NCE) of 27 and a maximum injection time of 200 ms and an automatic gain control (AGC) target of 50,000. Full MS data were acquired at 120,000 resolution in the orbitrap and HCD FTMS2 scans at a resolution of 30,000. Precursor ion isolation was carried out in the quadrupole using an isolation window of 2 m/z. The most intense precursor ion (z=2-4), as it had been previously determined for each peptide, was used for targeted analysis.

For the analysis and the generation of the precursor ion inclusion list, the unlabeled endogenous and the heavily labelled internal standard peptide variant were selected, additionally using a pre-defined retention time window during which the peptide was previously found to elute from the column. In case of Met-containing peptides, the unlabeled and isotopically labelled oxidized form was acquired and also the unlabeled reduced variant.

The inclusion list contained a final total of 36 precursor ions. Retention time frames over which a precursor was repeatedly triggered were determined in a fashion that a cycle time of 3 sec was not exceeded to allow for a minimum of 8 data points per peak.

Data analysis was carried out using Skyline software (MacLean et al., 2010) Peak integration, transition interferences and peak borders were adjusted and reviewed manually. A minimum of four transitions per precursor ion were considered and aₙ, bₙ, yₙ ions (where n≤2) excluded if applicable. Other filter criteria for successful detection included a maximum mass deviation of 10 ppm and spectral similarity of the light and the isotopically labelled peptide form, expressed as the library dot product with a minimum of 0.9. Precursor ion data were imported for further validation, however, not further used for quantitative analysis due to lack of specificity.

Total peptide intensity was calculated by summation of total fragment ion intensity of the endogenous light form divided by total fragment ion intensity of the isotopically labelled internal standard. Subsequent data processing was carried out using an in-house built script.

In brief, utilizing a previously acquired peptide-centric calibration curve which was constructed after digestion of refolded HLA-A*02:01/β2m monomer titration series in HLA-negative yeast lysate, each peptide-centric ratio was first transformed into a peptide concentration per total protein, expressed as fmol μg⁻¹.

The sample-specific HLA allotype composition of cell line MUTZ-3 was determined using RNAseq data followed by an in silico calculation performed on the TRON. Each sample non-HLA-A*02:01 allotype protein sequence present in MUTZ-3 (A*03:01, B*44:02, C*04:01, C*07:04) was now screened for the occurrence of any of the nine analyzed HLA-A*02:01 peptides (Table 1) and assigned accordingly for allotype-specific peptide groups.

Since β2m does not show any sequence polymorphisms but is rather highly conserved, both respective peptides (SEQ ID NO 11 & SEQ ID NO 12) were merged without any further sample-specific typing review.

Sample-dependent HLA allotype composition combined with in silico tryptic digestion and blasting versus SEQ ID NO 01 to SEQ ID NO 10 ultimately allowed to cluster the nine analyzed HLA-A*02:01 (SEQ ID NO 7 was left out for reasons not to be discussed here) peptides into various subgroups, depending on their matching HLA allotypes within the sample.

As an example, in MUTZ-3, only peptides SEQ ID NO 4, 6 & 8 were exclusive to HLA-A*02:01 whereas e.g. SEQ ID NO 1, 3 & 5 additionally matched to HLA-A*03:01 and are thus to be excluded from analysis of HLA-A*02:01. This yielded an absolute abundance of 64.7 fmol μg⁻¹ β2m and 12.9 fmol μg⁻¹ HLA-A*02:01 in MUTZ-3 cell lysate, both at a standard deviation below 20%.

Differential quantification of HLA-A*02:01 vs. [HLA-A*02:01+HLA-A*03:01; 17.8 fmol μg⁻¹] additionally allows to gain indirect insight into total abundance of HLA-A*03:01 in MUTZ-3 and was found to be 17.8-12.9 fmol μg⁻¹=4.9 fmol μg⁻¹. Likewise, analysis of HLA-C*07:04 protein levels only provided levels of 0.8 fmol μg⁻¹, transforming to a difference of HLA-C*07:04 to HLA-A*02:01 levels of ˜10-fold in MUTZ-3.

Further inclusion of protein concentration and total lysate volume yielded the total amount of peptide per cell lysate as also shown in Equation 2. By further taking the sample cell count into consideration, which was found be to at 500×10⁶ cells, protein copies per cell were finally obtained. In MUTZ-3, these were found to be 5.6×10⁶ for β2m and 1.1×10⁶ molecules for HLA-A*02:01. The difference between β2m and HLA-A*02:01 total protein abundance was found to be ˜5-fold in MUTZ-3.

### Example 2

As a biological source of MHC proteins in primary, non-cultured tissues, a human hepatocellular carcinoma sample (from here on depicted as “HCC-1”) was used.

A total of 0.68 g tumor tissue collected at University Hospital Tuebingen was subjected to cell lysis in a CHAPS detergent-containing buffer and homogenized assisted by sonification. Insoluble compounds were removed by ultracentrifugation and the cleared lysate was stored at −80° C. until further processing.

Prior to further downstream analysis, the protein concentration in the cleared lysate was determined using BCA assay. A titration series of bovine serum albumin in 50 mM ammonium bicarbonate was used as a calibration curve to calculate total protein concentration in the cell lysate. The protein concentration in the cleared lysate was found to be at 18.9 μg μL⁻¹.

The corresponding sample cell count was determined based on the quantification of total DNA content within the sample. For respective DNA isolation, an aliquot of the homogenized, non-centrifuged cell lysate was used. In brief, DNA was isolated and quantified using the fluorometric Qubit Assay (Thermo Fisher Scientific). The cell count was interpolated from DNA content using a titration series of peripheral blood mononuclear cells of known cell count. Prior to sample digestion, the internal standard mix containing the relevant overhang peptides as shown in Table 1 (and also Tables 2-4) at a stock concentration of 25 pmol μL⁻¹ was diluted to 100 fmol μ⁻¹ using 50 mM ammonium bicarbonate as a diluent.

Subsequently, proteolytic digestion was initiated by adding 150 μSMART Digest buffer, 10 μL 100 fmol μL⁻¹ internal standard mix, 20 μg total protein from HCC-1 cell lysate (i.e. 1.1 μL lysate at 18.9 μg μL⁻¹ as determined previously) to the corresponding SMART Digest trypsin aliquot vial. Finally, H₂O_{dd} was added to a final total volume of 200 μL and the reaction tube was stirred for 3 sec.

The sample was transferred to a pre-heated heating block and efficient proteolytic digestion initiated by incubation at 70° C. for 90 min at 1,400 rpm. In order to denature trypsin afterwards and thus irreversibly stop the proteolytic digestion, TFA was added to the reaction tube at a final concentration of 0.5%, which lowered the pH to <3.

For sample clean-up (i.e. removal of salts and remaining high-molecular weight compounds such as trypsin beads) prior to LC-MS/MS analysis, C18 reverse-phase solid-phase extraction was used employing 0.1% TFA as a wash solvent of the C18-bound peptides. After peptide elution using 70% ACN, the sample was lyophilized to complete dryness and subsequently reconstituted in 5% FA at a concentration of 500 ng μL⁻¹.

The peptide mixture was then subjected to liquid chromatography coupled to mass spectrometry (LC-MS/MS) using a nanoACQUITY UPLC system (Waters) coupled online to an Orbitrap Fusion™ Tribrid™ mass spectrometer (Thermo Fisher Scientific) at a flow rate of 300 n1 min⁻¹. Data were acquired in three technical replicates and a total of 250 ng sample was loaded onto the column per LC-MS/MS run.

The mass spectrometer was operated in scheduled parallel reaction monitoring (sPRM) mode to allow for the targeted analysis of the pre-selected probe set. Nano-flow sPRM assays were performed using a 42 min three-step linear, binary gradient consisting of solvent A (0.1% FA in H₂O) and solvent B (0.1% FA in ACN). For successful peptide ion dissociation, higher-energy collisional dissociation (HCD) was employed at a normalized collision energy (NCE) of 27 and a maximum injection time of 200 ms and an automatic gain control (AGC) target of 50,000. Full MS data were acquired at 120,000 resolution in the orbitrap and HCD FTMS2 scans at a resolution of 30,000. Precursor ion isolation was carried out in the quadrupole using an isolation window of 2bm/z. The most intense precursor ion (z=2-4), as it had been previously determined for each peptide, was used for targeted analysis.

For the analysis and the generation of the precursor ion inclusion list, the unlabelled endogenous and the heavily labelled internal standard peptide variant were selected, additionally using a pre-defined retention time window during which the peptide was previously found to elute from the column. In case of Met-containing peptides, the unlabelled and isotopically labelled oxidized form was acquired and also the unlabelled reduced variant.

The inclusion list contained a final total of 36 precursor ions. Retention time frames over which a precursor was repeatedly triggered were determined in a fashion that a cycle time of 3 sec was not exceeded to allow for a minimum of 8 data points per peak.

Data analysis was carried out using Skyline software (MacLean et al., 2010). Peak integration, transition interferences and peak borders were adjusted and reviewed manually. A minimum of four transitions per precursor ion were considered and aₙ, bₙ, yₙ ions (where n≤2) excluded if applicable. Other filter criteria for successful detection included a maximum mass deviation of 10 ppm and spectral similarity of the light and the isotopically labelled peptide form, expressed as the library dot product with a minimum of 0.9. Precursor ion data were imported for further validation, however, not further used for quantitative analysis due to lack of specificity.

Total peptide intensity was calculated by summation of total fragment ion intensity of the endogenous light form divided by total fragment ion intensity of the isotopically labelled internal standard. Subsequent data processing was carried out using an in-house built script.

In brief, utilizing a previously acquired peptide-centric calibration curve which was constructed after digestion of refolded HLA-A*02:01/β2m monomer titration series in HLA-negative yeast lysate, each peptide-centric ratio was first transformed into a peptide concentration per total protein, expressed as fmol Results are shown in FIG. 7.

The sample-specific HLA allotype composition of non-cultured primary tissue sample HCC-1 was determined using RNAseq data followed by an in silico calculation performed on the TRON server (Seq2HLA algorithm; typing depicted in FIG. 9B). Each sample non-HLA-A*02:01 allotype protein sequence present in HCC-1 (A*23:01, B*15:01, B*44:03, C*01:02, C*04:01) was now screened for the occurrence of any of the nine analyzed HLA-A*02:01 peptides (Table 1) and assigned accordingly for allotype-specific peptide groups (FIG. 9B lower table and C).

Since β2m does not show any sequence polymorphisms but is rather highly conserved, both respective peptides (SEQ ID NO 11 & SEQ ID NO 12) were merged without any further sample-specific typing review.

Sample-dependent HLA allotype composition combined with in silico tryptic digestion and blasting versus SEQ ID NO 1 to SEQ ID NO 10 ultimately allowed to cluster the nine analyzed HLA-A*02:01 peptides into various subgroups, depending on their matching HLA allotypes within the sample.

Here, only peptides SEQ ID NO 4, 5, 8 & 10 were exclusive to HLA-A*02:01 whereas e.g. SEQ ID NO 3, 6 & 9 additionally matched to HLA-A*23:01 and are thus to be excluded from analysis of HLA-A*02:01. This yielded an absolute abundance of 35.5 fmol μg⁻¹ β2m and 7.0 fmol μg⁻¹ HLA-A*02:01 in HCC-1 cell lysate, both at a standard deviation below 15%.

Differential quantification of HLA-A*02:01 vs. [HLA-A*02:01+HLA-A*23:01; 13.2 fmol μg⁻¹] additionally allows to gain indirect insight into total abundance of HLA-A*23:01 in HCC-1 and was found to be 13.2-7.0 fmol μg⁻¹=6.2 fmol μg⁻¹. Likewise, analysis of HLA-C*01:02 protein levels only provided levels of 0.7 fmol μg⁻¹, transforming to a difference of HLA-C*01:02 to HLA-A*02:01 levels of ˜10-fold in HCC-1. This observation confirms findings as shown in example 1 with regard to the relative expression of HLA-C in comparison to HLA-A, which was found to be 10-fold in both cases.

Further inclusion of protein concentration and total lysate volume yielded the total amount of peptide per cell lysate as also shown in Equation 2. By further taking the sample cell count into consideration, which was calculated to be 240×10⁶ cells, protein copies per cell were finally obtained. In HCC-1, these were found to be 5.6×10⁶ for β2m and 1.1×10⁶ molecules for HLA-A*02:01 and coincidentally match copy numbers as shown in example 1. The difference between β2 and HLA-A*02:01 total protein abundance was thus found to be ˜5-fold in HCC-1 as well.

### Example 3

As a biological source of MHC proteins in primary, non-cultured tissues, a human small cell carcinoma of the lung (from here on depicted as “SCLC-1”) was used. A total of 0.61 g tumor tissue provided by Asterand Bioscience was subjected to cell lysis in a CHAPS detergent-containing buffer and homogenized assisted by sonification. Insoluble compounds were removed by ultracentrifugation and the cleared lysate was stored at −80° C. until further processing. Prior to further downstream analysis, the protein concentration in the cleared lysate was determined using bicinchoninic acid (BCA) assay. A titration series of bovine serum albumin in 50 mM ammonium bicarbonate was used as a calibration curve to calculate total protein concentration in the cell lysate. The protein concentration in the cleared lysate was found to be at 12.4 μg μL⁻¹. The corresponding sample cell count was determined based on the reverse correlation of its tissue weight via a tissue weight-based regression curve correlated with a cohort of data, for which cell counts have been previously determined via a fluorescence-based DNA quantification.

Proteolytic processing was initiated by adding 20 μg total protein from SCLC-1 cell lysate (i.e. 1.6 μL lysate at 12.4 μg μL⁻¹ as determined previously) to an reaction vial. For reduction and alkylation of Cysteine disulfide bonds, Tris(2-carboxyethyl)phosphine hydrochloride (TCEP) and chloro-acetamide (CAA) were added to a final concentration of 10 mM and 40 mM, respectively, followed by incubation at 70° C. for 10 min. Subsequently, for protein enrichment and purification, 200 μg carboxylated paramagnetic beads were added.

Protein binding to the beads was induced by addition of ACN to a final concentration of 50% (V/V) followed by incubation for 10 min at 24° C. and stirring at 1,000 rpm. The sample was placed at a magnetic separation stand and the supernatant was removed followed by addition of 80% EtOH for detergent removal.

The supernatant was removed and EtOH was added followed by removal of the supernatant on the magnetic separation stand. The internal standard mix containing relevant overhang peptides as shown in Tables 1 to 4 was diluted to 100 fmol μ⁻¹ using 50 mM ammonium bicarbonate as diluent and subsequently 10 μL of diluted internal standard mix were added to the reaction vial.

For proteolytic digestion, 100 μL AmBic (100 mM) and 2 μg trypsin/LysC (Promega) were added accompanied by addition of ProteaseMax (Promega) to a final concentration of 0.03%. The sample was subsequently incubated for 18 h at 37° C. and 1,000 rpm.

After completion of proteolytic digestion, the sample was placed on a magnetic separation stand and the supernatant containing the peptide mixture was transferred to a new reaction vial.

The peptide mixture was then subjected to liquid chromatography coupled to mass spectrometry (LC-MS/MS) using an EvoSep One (Evosep) coupled online to an Orbitrap Eclipse™ mass spectrometer (Thermo Fisher Scientific). Data were acquired in two technical replicates and a total of 500 ng sample was loaded onto the column per LC-MS/MS run. The mass spectrometer was operated in scheduled parallel reaction monitoring (sPRM) mode to allow for the targeted analysis of the pre-selected probe set. Nano-flow sPRM assays were performed using a standardized pre-formed 44 min binary gradient consisting of solvent A (0.1% FA in H₂O) and solvent B (0.1% FA in ACN). For successful peptide ion dissociation, higher-energy collisional dissociation (HCD) was employed at a normalized collision energy (NCE) of 27 and a maximum injection time of 54 ms and an automatic gain control (AGC) target of 1,000%. Full MS data were acquired at 120,000 resolution in the orbitrap and HCD FTMS2 scans at a resolution of 30,000. Precursor ion isolation was carried out in the quadrupole using an isolation window of 1.6 m/z. The most intense precursor ion (z=2-4), as it had been previously determined for each peptide, was used for targeted analysis. For the analysis and the generation of the precursor ion inclusion list, the unlabelled endogenous and the heavily labelled internal standard peptide variant were selected, additionally using a predefined retention time window during which the peptide was previously found to elute from the column. In case of Met-containing peptides, the unlabelled and isotopically labelled oxidized form was acquired and also the unlabelled reduced variant. The inclusion list contained a final total of 66 precursor ions. Retention time frames over which a precursor was repeatedly triggered were determined in a fashion that a cycle time of 3 sec was not exceeded to allow for a minimum of 7 data points per peak. Data analysis was carried out using Skyline software (MacLean et al., 2010). Peak integration, transition interferences and peak borders were adjusted and reviewed manually. A minimum of four transitions per precursor ion were considered and aₙ, bₙ, yₙ ions (where n≤2) excluded if applicable. Other filter criteria for successful detection included a maximum mass deviation of 10 ppm and spectral similarity of the light and the isotopically labelled peptide form, expressed as the library dot product with a minimum of 0.9. Precursor ion data were imported for further validation, however, not further used for quantitative analysis due to lack of specificity.

Total peptide intensity was calculated by summation of total fragment ion intensity of the endogenous light form divided by total fragment ion intensity of the isotopically labelled internal standard. Subsequent data processing was carried out using an in-house built script. In brief, utilizing previously acquired peptide-centric calibration curves which were either constructed after digestion of refolded HLA-A*02:01/β2m monomer or HLA-B*07:02/β2m monomer titration series in HLA-negative yeast lysate, each peptide-centric ratio was first transformed into a peptide concentration per total protein, expressed as fmol μg⁻¹. Results are shown in FIG. 11. The sample-specific HLA allotype composition of non-cultured primary tissue sample SCLC-1 was determined using RNAseq data followed by an in silico calculation performed on the TRON server (Seq2HLA algorithm; typing depicted in FIG. 11). Each sample non-HLA A*02:01/B*07:02 allotype protein sequence present in SCLC-1 (A*11:01, B*35:01, C*04:01 & C*07:02) was now screened for the occurrence of any of the nine analyzed HLA-A*02:01 peptides (Table 1) or eight B*07:02-specific peptides (Table 4) and assigned accordingly for allotype-specific peptide groups (FIG. 11B lower table and 11C). Since β2m does not show any sequence polymorphisms but is rather highly conserved, both respective peptides (SEQ ID NO 11 & SEQ ID NO 12) were merged without any further sample-specific typing review.

Sample-dependent HLA allotype composition combined with in silico tryptic digestion and blasting versus respective SEQ IDs ultimately allowed to cluster the nine analyzed HLA-A*02:01 and eight B*07:02 peptides into various subgroups, depending on their matching HLA allotypes within the sample.

Here, only peptides SEQ ID NO 4, 6 & 8 were exclusive to HLA-A*02:01 whereas e.g. SEQ ID NO 3 & 5 additionally matched to HLA-A*11:01 and are thus to be excluded from quantification of HLA-A*02:01. SEQ ID 53 peptide uniquely matched to B*07:02 here. This yielded an absolute abundance of 41.6 fmol μg⁻¹ β2 m, 19.4 fmol μg⁻¹ HLA-A*02:01 and 11.4 fmol μg⁻¹ HLA-B*07:02 in SCLC-1 cell lysate, all three calculated at a standard deviation below 25%.

### Example 4: Histone-Derived Cell Count

Histones are highly basic proteins found in eukaryotic cell nuclei that pack and order the DNA into structural units called nucleosomes. Histones are the chief protein components of chromatin, acting as spools around which DNA winds, and playing a role in gene regulation. Because, in a diploid cell, the amount of DNA is constant, the amount of histone is also constant. Five major families of histones exist: H1/H5, H2A, H2B, H3, and H4. Histones H2A, H2B, H3, and H4 are known as the core histones, while histones H1/H5 are known as the linker histones.

According to one embodiment of the method according to the invention, at least one protein the abundance of which is roughly proportional to the total number of cells in the sample is a histone, e.g., histone H2A, histone H2B, or histone H4. Histone H2A (UniProt ID B2R5B3) is one of the main histone proteins involved in the structure of chromatin in eukaryotic cells. H2A utilizes a protein fold known as the “histone fold”. The 25 histone fold is a three-helix core domain that is connected by two loops. This connection forms a “handshake arrangement”. Most notably, this is termed the helix-turn-helix motif, which allows for dimerization with H2B. Histone H2B (UniProt ID B4DR52) is another one of the main histone proteins involved in the structure of chromatin in eukaryotic cells. Two copies of histone H2B come together with two copies each of histone H2A, histone H3, and histone H4 to form the octamer core of the nucleosome to give structure to DNA. Histone H4 (UniProt ID Q6B823) is yet another one of the main histone proteins involved in the structure of chromatin in eukaryotic cells. Histone proteins H3 and H4 bind to form a H3-H4 dimer, two of these H3-H4 dimers combine to form a tetramer. This tetramer further combines with two H2a-H2b dimers to form the compact Histone octamer core. Generally, the abundance of histones, is due to their DNA-binding capacity, proportional to the total number of cells in the sample. Quantifying histones in a sample hence provides an estimate of the total number of cells comprised therein.

For this purpose, according to one embodiment, a calibration curve is established by titration of one or more cells vs. a histone-based signal, as obtained by the spectrometry methods disclosed herein. More precisely, the ratio of endogenous histone peptides obtained by tryptic digestion versus their heavy isotope-labelled internal standard peptides is determined.

The following peptide sequences can be used for the quantification of the different histones

| | |
|---|---|
| Histone H2A | SEQ ID NO: 13/30 |
| Histone H2B | SEQ ID NO: 14/31 |
| Histone H4 | SEQ ID NO: 15/32 |
| Histone H4 | SEQ ID NO: 16/33 |
| Histone H4 | SEQ ID NO: 17/34 |

Calibration by Means of PBMC

In order to determine a calibration curve of histone peptides, it is critical that the calibrant has a defined diploid cell count. Therefore, peripheral blood mononuclear cells were chosen as a calibrant since their cell count can be easily assessed via manual cell counting. For the acquisition of the calibration curve, PBMCs were isolated from whole blood and subsequently split into aliquots of 5 Mio, 10 Mio, 50 Mio, 100 Mio, 200 Mio, and 500 Mio cells (see FIGS. 12A and 13). The resulting cell pellets were subjected to cell lysis in a CHAPS detergent-containing buffer and homogenized assisted by sonification. Insoluble compounds were removed by ultracentrifugation and the cleared lysate was stored at −80° C. until further processing. Prior to further downstream analysis, the protein concentration in the cleared lysate was determined using BCA assay. A titration series of bovine serum albumin in 50 mM ammonium bicarbonate was used as a calibration curve to calculate total protein concentration in the cell lysate. The protein concentration in the cleared lysate were found to be as follows:

| Cell count | Protein concentration [μg μL−¹] |
|---|---|
| 1 mio | 0.12 |
| 5 Mio | 0.33 |
| 10 Mio | 0.78 |
| 50 Mio | 2.35 |
| 100 Mio | 3.23 |
| 200 Mio | 4.61 |
| 250 Mio | 4.33 |
| 350 Mio | 3.79 |
| 500 Mio | 5.21 |

Subsequently, proteolytic digestion was initiated by adding 150 μL SMART Digest buffer, 10 μL 100 fmol μL⁻¹ internal standard mix, 20 μg total protein from the respective PBMC lysate to the corresponding SMART Digest trypsin aliquot vial.

Finally, H₂O_{dd} was added to a final total volume of 200 μL and the reaction tube was stirred for 3 sec. The sample was transferred to a pre-heated heating block and efficient proteolytic digestion was initiated by incubation at 70° C. for 90 min at 1,400 rpm. In order to denature trypsin afterwards and thus irreversibly stop the proteolytic digestion, TFA was added to the reaction tube at a final concentration of 0.5%, which lowered the pH to <3.

For sample clean-up (i.e. removal of salts and remaining high-molecular weight compounds such as trypsin beads) prior to LC-MS/MS analysis, C18 reverse-phase solid-phase extraction was used employing 0.1% TFA as a wash solvent of the C18-bound peptides. After peptide elution using 70% ACN, the sample was lyophilized to complete dryness and subsequently reconstituted in 5% FA at a concentration of 500 ng μL⁻¹.

The peptide mixture was then subjected to liquid chromatography coupled to mass spectrometry (LC-MS/MS) using a nanoACQUITY UPLC system (Waters) coupled online to an Orbitrap Fusion™ Tribrid™ mass spectrometer (Thermo Fisher Scientific) at a flow rate of 300 nl min⁻¹. Data were acquired in three technical replicates and a total of 250 ng sample was loaded onto the column per LC-MS/MS run.

The mass spectrometer was operated in scheduled parallel reaction monitoring (sPRM) mode to allow for the targeted analysis of the pre-selected probe set. Nano-flow sPRM assays were performed using a 42 min three-step linear, binary gradient consisting of solvent A (0.1% FA in H₂O) and solvent B (0.1% FA in ACN). For successful peptide ion dissociation, higher-energy collisional dissociation (HCD) was employed at a normalized collision energy (NCE) of 27 and a maximum injection time of 200 ms and an automatic gain control (AGC) target of 50,000. Full MS data were acquired at 120,000 resolution in the orbitrap and HCD FTMS2 scans at a resolution of 30,000. Precursor ion isolation was carried out in the quadrupole using an isolation window of 2 m/z. The most intense precursor ion (z=2−4), as it had been previously determined for each histone peptide, was used for targeted analysis.

For the analysis and the generation of the precursor ion inclusion list, the unlabelled endogenous and the heavily labelled internal standard peptide variant were selected, additionally using a predefined retention time window during which the peptide was previously found to elute from the column. In case of Met-containing peptides, the unlabelled and isotopically labelled oxidized form was acquired and also the unlabelled reduced variant.

The inclusion list contained a final total of 36 precursor ions. Retention time frames over which a precursor was repeatedly triggered were determined in a fashion that a cycle time of 3 sec was not exceeded to allow for a minimum of 8 data points per peak.

Data analysis was carried out using Skyline software (MacLean et al., 2010). Peak integration, transition interferences and peak borders were adjusted and reviewed manually. A minimum of four transitions per precursor ion were considered and aₙ, bₙ, yₙ ions (where n≤2) excluded if applicable. Other filter criteria for successful detection included a maximum mass deviation of 10 ppm and spectral similarity of the unlabelled (“light”) and the isotopically labelled (“heavy”) peptide form, expressed as the library dot product with a minimum of 0.9. Precursor ion data were imported for further validation, however, not further used for quantitative analysis due to lack of specificity. Total peptide intensity was calculated by summation of total fragment ion intensity of the endogenous light form divided by total fragment ion intensity of the isotopically labelled internal standard. Subsequent data processing was carried out using an in-house built script.

### Transfer to Other Tissues

Tissue samples taken from spleen, cartilage, adipose tissue, heart, kidney and hepatocellular carcinoma (HCC) were treated in like fashion to determine the histone content. Based on the calibration curves obtained with PBMC (see e.g. FIG. 13), total cell count was then calculated. Results are shown in FIG. 12B

It is critical to not just take the number of histones (as determined via MS analysis of the sample and using and spiking in an internal standard for absolute quantification, as e.g disclosed in Edfors et al. (2016)) but to use this histone amount, consider it as some sort of ‘arbitrary value’ and correlate it with the actual cell count of the sample. By doing this, we account for protein/histone losses during sample processing and also for any unbound histones which may be present in the nucleus. The titration series of PBMCs (either in a histone-negative protein matrix, such as yeast, or just as pure PBMCs) gives a calibration curve. The total number of the respective histones is hereby calculated.

In FIGS. 12A and B some examples of different healthy and cancerous primary tissues are shown for which we have calculated the total number of histones via the spiked-in standards and translated it back into the total number of cells using the previously acquired calibration curve. H2ATR-001 is SEQ ID NO: 13, H2BTR-001 is SEQ ID NO: 14, H4TR-001 is SEQ ID NO: 15 and H4TR-002 is SEQ ID NO: 16. Note that, yet, in the method, the peptides that were spiked in comprised N- and C-terminal overhangs for tryptic digestion (H2ATR-001: SEQ ID NO: 30, H2BTR-001: SEQ ID NO: 31, H4TR-001: SEQ ID NO: 32 and H4TR-002: SEQ ID NO: 33).

In FIG. 13 histone-based calibration curves are shown that have been established using PBMC cell count.

### Example 5

As discussed, further sample peptide analogues were established to quantify, inter alia, the allotypes HLA-A*01:01; HLA-A*03:01; HLA-A*24:02; HLA-B*07:02; HLA-B*08:01; HLA-B*44:02 and HLA-B*44:03. These peptides are shown in Table 4 ctd′.

Based on the sample peptide analogues disclosed in FIGS. 4 and 10, and also in Tables 1 and 4, the skilled person can assemble sets of sample peptide analogues for the quantification of different HLA allotypes in a sample either individually, or simultaneously.

In order to allow absolute quantification, the sample peptide analogues of FIG. 4 that are derived from ß2 microglobulin and/or the histones (see also tables 2 and 3) can be added to the set of sample peptide analogues.

Hence, FIGS. 4 and 10, together with tables 1-4, provide a toolbox that allows the relative of absolute quantification of one or more HLA allotypes in a given sample.

### REFERENCES

The disclosures of these documents are herein incorporated by reference in their entireties.
Apps R, Meng Z, Del Prete G Q, Lifson J D, Zhou M, Carrington M (2015). Relative expression levels of the HLA class-I proteins in normal and HIV-infected cells. J Immunol 194, 3594-3600.
Basu D, Horvath S, O'Mara L, Donermeyer D, Allen P M (2001). Two MHC surface amino acid differences distinguish foreign peptide recognition from autoantigen specificity. J Immunol 166, 4005-4011.
Boniface J J, Davis M M (1995). T-cell recognition of antigen. A process controlled by transient intermolecular interactions. Ann N Y Acad Sci 766, 62-69.
Caillat-Zucman S (2009). Molecular mechanisms of HLA association with autoimmune diseases. Tissue Antigens 73, 1-8.

Caron E, Kowalewski D J, Chiek Koh C, Sturm T, Schuster H, Aebersold R (2015). Analysis of Major Histocompatibility Complex (MHC) Immunopeptidomes Using Mass Spectrometry. Mol Cell Proteomics. 14(12), 3105-17.
Chang A Y, Gejman R S, Brea E J, Oh C Y, Mathias M D, Pankov D, Casey E, Dao T, Scheinberg D A (2016). Opportunities and challenges for TCR mimic antibodies in cancer therapy. Expert Opin Biol Ther 16, 979-987.
Chowell D, Morris L G T, Grigg C M, Weber J K, Samstein R M, Makarov V, Kuo F, Kendall S M, Requena D, Riaz N, et al. (2018). Patient HLA class I genotype influences cancer response to checkpoint blockade immunotherapy. Science 359, 582-587.
Coley W B (1991). The treatment of malignant tumors by repeated inoculations of erysipelas. With a report of ten original cases. 1893. Clin Orthop Relat Res, 3-11.
Coulie P G, Van Den Eynde B J, van der Bruggen P, Boon T (2014). Tumour antigens recognized by T lymphocytes: at the core of cancer immunotherapy. Nat Rev Cancer 14, 135-146.
Dahan R, Reiter Y (2012). T-cell-receptor-like antibodies—generation, function and applications. Expert Rev Mol Med 14, e6.
Dao T, Pankov D, Scott A, Korontsvit T, Zakhaleva V, Xu Y, Xiang J, Yan S, de Morais Guerreiro M D, Veomett N, et al. (2015). Therapeutic bispecific T-cell engager antibody targeting the intracellular oncoprotein WT1. Nat Biotechnol 33, 1079-1086.
Davenport A J, Cross R S, Watson K A, Liao Y, Shi W, Prince H M, Beavis P A, Trapani J A, Kershaw M H, Ritchie D S, et al. (2018). Chimeric antigen receptor T cells form nonclassical and potent immune synapses driving rapid cytotoxicity. Proc Natl Acad Sci USA 115, E2068-E2076.
Delves P J, Roitt I M (2000). The immune system. Second of two parts. N Engl J Med 343, 108-117.
Edfors F, Danielsson F, Hallström B M, Käll L, Lundberg E, Pontén F, Forsström B, Uhlén M (2016). Gene-specific correlation of RNA and protein levels in human cells and tissues. Mol Syst Biol. 12(10), 883.
Falk K, Rotzschke 0, Stevanovic S, Jung G, Rammensee H G (1991). Allele-specific motifs revealed by sequencing of self-peptides eluted from MHC molecules. Nature 351, 290-296.
Felix N J, Allen P M (2007). Specificity of T-cell alloreactivity. Nat Rev Immunol 7, 942-953.
Freudenmann L K, Marcu A, Stevanovic S (2018). Mapping the tumour human leukocyte antigen (HLA) ligandome by mass spectrometry. Immunology 154, 331-345.
Gao G F, Willcox B E, Wyer J R, Boulter J M, O'Callaghan C A, Maenaka K, Stuart D I, Jones E Y, Van Der Merwe P A, Bell J I, et al. (2000). Classical and nonclassical class I major histocompatibility complex molecules exhibit subtle conformational differences that affect binding to CD8alphaalpha. J Biol Chem 275, 15232-15238.
Goldberg A C, Rizzo L V (2015a). MHC structure and function—antigen presentation. Part 1. Einstein (Sao Paulo) 13, 153-156.
Goldberg A C, Rizzo L V (2015b). MHC structure and function—antigen presentation. Part 2. Einstein (Sao Paulo) 13, 157-162.
Gonzalez-Galarza F F, Takeshita L Y, Santos E J, Kempson F, Maia M H, da Silva A L, Teles e Silva A L, Ghattaoraya G S, Alfirevic A, Jones A R, et al. (2015). Allele frequency net 2015 update: new features for HLA epitopes, M R and disease and HLA adverse drug reaction associations. Nucleic Acids Res 43, D784-788.
Gruen J R, Weissman S M (1997). Evolving views of the major histocompatibility complex. Blood 90, 4252-4265.
Guo H C, Jardetzky T S, Garrett T P, Lane W S, Strominger J L, Wiley D C (1992). Different length peptides bind to HLA-Aw68 similarly at their ends but bulge out in the middle. Nature 360, 364-366.
He Q, Liu Z, Liu Z, Lai Y, Zhou X, Weng J (2019). TCR-like antibodies in cancer immunotherapy. J Hematol Oncol 12, 99.
Hilf N, Kuttruff-Coqui S, Frenzel K, Bukur V, Stevanovic S, Gouttefangeas C, Platten M, Tabatabai G, Dutoit V, van der Burg S H, et al. (2019). Actively personalized vaccination trial for newly diagnosed glioblastoma. Nature 565, 240-245.
Hill A V, Allsopp C E, Kwiatkowski D, Anstey N M, Twumasi P, Rowe P A, Bennett S, Brewster D, McMichael A J, Greenwood B M (1991). Common west African HLA antigens are associated with protection from severe malaria. Nature 352, 595-600.
Kirner A, Mayer-Mokler A, Reinhardt C (2014). IMA901: a multi-peptide cancer vaccine for treatment of renal cell cancer. Hum Vaccin Immunother 10, 3179-3189.
Kuhn N F, Purdon T J, van Leeuwen D G, Lopez A V, Curran K J, Daniyan A F, Brentjens R J (2019). CD40 Ligand-Modified Chimeric Antigen Receptor T Cells Enhance Antitumor Function by Eliciting an Endogenous Antitumor Response. Cancer Cell 35, 473-488 e476.
Lustgarten J, Waks T, Eshhar Z (1991). CD4 and CD8 accessory molecules function through interactions with major histocompatibility complex molecules which are not directly associated with the T cell receptor-antigen complex. Eur J Immunol 21, 2507-2515.
MacLean B, Tomazela D M, Shulman N, Chambers M, Finney G L, Frewen B, Kern R, Tabb D L, Liebler D C, MacCoss M J (2010). Skyline: an open source document editor for creating and analyzing targeted proteomics experiments. Bioinformatics 26, 966-968.
Madden D R, Garboczi D N, Wiley D C (1993). The antigenic identity of peptide-MHC complexes: a comparison of the conformations of five viral peptides presented by HLA-A2. Cell 75, 693-708.
Molenkamp B G, Vuylsteke R J, van Leeuwen P A, Meijer S, Vos W, Wijnands P G, Scheper R J, de Gruijl T D (2005). Matched skin and sentinel lymph node samples of melanoma patients reveal exclusive migration of mature dendritic cells. Am J Pathol 167, 1301-1307.
Olson B J, Markwell J (2007). Assays for determination of protein concentration. Curr Protoc Protein Sci Chapter 3, Unit 3 4.
Rammensee H G (1995). Chemistry of peptides associated with MHC class I and class II molecules. Curr Opin Immunol 7, 85-96.
Rammensee H G, Friede T, Stevanoviic S (1995). MHC ligands and peptide motifs: first listing. Immunogenetics 41, 178-228.
Ray K, Marteyn B, Sansonetti P J, Tang C M (2009). Life on the inside: the intracellular lifestyle of cytosolic bacteria. Nat Rev Microbiol 7, 333-340.
Robinson J, Halliwell J A, Hayhurst J D, Flicek P, Parham P, Marsh S G (2015). The IPD and IMGT/HLA database: allele variant databases. Nucleic Acids Res 43, D423-431.
Rock K L, Shen L (2005). Cross-presentation: underlying mechanisms and role in immune surveillance. Immunol Rev 207, 166-183.
Rosenberg S A, Yang J C, Sherry R M, Kammula U S, Hughes M S, Phan G Q, Citrin D E, Restifo N P, Robbins P F, Wunderlich J R, et al. (2011). Durable complete responses in heavily pretreated patients with metastatic melanoma using T-cell transfer immunotherapy. Clin Cancer Res 17, 4550-4557.
Schwanhausser B, Busse D, Li N, Dittmar G, Schuchhardt J, Wolf J, Chen W, Selbach M (2011). Global quantification of mammalian gene expression control. Nature 473, 337-342.
Schwanhausser B, Busse D, Li N, Dittmar G, Schuchhardt J, Wolf J, Chen W, Selbach M (2013). Corrigendum: Global quantification of mammalian gene expression control. Nature 495, 126-127.
Sharma P, Kranz D M (2016). Recent advances in T-cell engineering for use in immunotherapy. F1000Res 5.
Siller-Farfan J A, Dushek 0 (2018). Molecular mechanisms of T cell sensitivity to antigen. Immunol Rev 285, 194-205.
Stern L J, Wiley D C (1994). Antigenic peptide binding by class I and class II histocompatibility proteins. Structure 2, 245-251.
The International HIV Controllers Study, Pereyra F, Jia X, McLaren P J, Telenti A, de Bakker P I, Walker B D, Ripke S, Brumme C J, Pulit S L, et al. (2010). The major genetic determinants of HIV-1 control affect HLA class I peptide presentation. Science 330, 1551-1557.
Trachtenberg E, Korber B, Sollars C, Kepler T B, Hraber P T, Hayes E, Funkhouser R, Fugate M, Theiler J, Hsu Y S, et al. (2003). Advantage of rare HLA supertype in HIV disease progression. Nat Med 9, 928-935.
Urban J L, Schreiber H (1992). Tumor antigens. Annu Rev Immunol 10, 617-644.
van der Merwe P A, Dushek 0 (2011). Mechanisms for T cell receptor triggering. Nat Rev Immunol 11, 47-55.
Velcheti V, Schalper K (2016). Basic Overview of Current Immunotherapy Approaches in Cancer. Am Soc Clin Oncol Educ Book 35, 298-308.
Whitelegg A M, Oosten L E, Jordan S, Kester M, van Halteren A G, Madrigal J A, Goulmy E, Barber L D (2005). Investigation of peptide involvement in T cell allorecognition using recombinant HLA class I multimers. J Immunol 175, 1706-1714.
Yewdell J W (2003). Immunology. Hide and seek in the peptidome. Science 301, 1334-1335.
Yewdell J W, Anton L C, Bennink J R (1996). Defective ribosomal products (DRiPs): a major source of antigenic peptides for MHC class I molecules? J Immunol 157, 1823-1826.

### Sequences

The following sequences form part of the disclosure of the present application. A WIPO ST 25 compatible electronic sequence listing is provided with this application, too. For the avoidance of doubt, if discrepancies exist between the sequences in the following table and the electronic sequence listing, the sequences in this table shall be deemed to be the correct ones.

| | | | | |
|---|---|---|---|---|
| | which HLA | | | |
| SEQ | allotype or | | SEQ | Sequence with overhangs |
| ID | protein | Sequence w/o | ID | for tryptic digestion |
| NO | (example) | overhangs | NO | (“overhang peptides”) |

Table 1: Peptides for the quantification of HLA-A*02:01 and others

| | | | | |
|---|---|---|---|---|
| 1 | HLA-A*02:01; | YFFTSV*SRPGR | 18 | SMRYFFTSV*SRPGRGEP |
| | HLA-A*03:01 | | | |
| 2 | HLA-A*02:01 | FIAV*GYVDDTQFVR | 19 | EPRFIAV*GYVDDTQFVRFDS |
| 3 | HLA-A*02:01; | FDSDAASQ*R | 20 | FVRFDSDAASQ*RMEP |
| | HLA-A*23:01; | | | |
| | HLA-A*03:01 | | | |
| 4 | HLA-A*02:01 | APWIEQEGPEY*WDGETR | 21 | EPRAPWIEQEGPEY*WDGETRKVK |
| 5 | HLA-A*02:01; | VDLGTL*R | 22 | THRVDLGTL*RGYY |
| | HLA-A*03:01 | | | |
| 6 | HLA-A*02:01; | GYHQYAYDGK* | 23 | FLRGYHQYAYDGK*DYI |
| | HLA-A*23:01 | | | |
| 7 | HLA-A*02:01 | SWTAADBAAQTTK* | 24 | DLRWTAAD [symbol] AAQTTK*HKW |
| 8 | HLA-A*02:01 | WEAAHVAEQL*R | 25 | KHKWEAAHVAEQL*RAYL |
| 9 | HLA-A*02:01; | DGEDQTQDTELVETRPAGDG | 26 | WQRDGEDQTQDTELVETRPAGDGTF*QKWAA |
| | HLA-A*23:01 | TF*QK | | |
| 10 | HLA-A*02:01 | WAAVVVPSGQEQ*R | 27 | FQKWAAVVVPSGQEQ*RYTC |

Table 2: Peptides for the quantification of β2m

| | | | | |
|---|---|---|---|---|
| 11 | β2m | VEHSDL*SFSK | 28 | IEKVEHSDL*SFSKDWS |
| 12 | β2m | VNHVTL*SQPK | 29 | ACRVNHVTL*SQPKIVK |

Table 3: Peptides for the quantification of Histones

| | | | | |
|---|---|---|---|---|
| 13 | derived from | AGL*QFPVGR | 30 | SSRAGL*QFPVGRVHR |
| | Histone H2A | | | |
| 14 | derived from | LLLPGEL*AK | 31 | AVRLLLPGEL*AKHAV |
| | Histone H2B | | | |
| 15 | derived from | ISGL*IYEETR | 32 | VKRISGL*IYEETRGVL |
| | Histone H4 | | | |
| 16 | derived from | VFL*ENVIR | 33 | VLKVFL*ENVIRDAV |
| | Histone H4 | | | |
| 17 | derived from | TVTABDVVYAL*K | 34 | KRKTVTABDVVYAL*KRQG |
| | Histone H4 | | | |

Table 4: Peptides for the quantification of other HLA allotypes

| | | | | |
|---|---|---|---|---|
| 44 | HLA-A*01:01 | ANL*GTLR | 63 | TDRANL*GTLRGYY |
| 45 | HLA-A*24:02 | APWIEQEGPEY*WDEETGK | 64 | EPRAPWIEQEGPEY*WDEETGKVKA |
| 46 | HLA-B*07:02; | AP*WIEQEGPEYWDR | 65 | EPRAP*WIEQEGPEYWDRNTQ |
| | HLA-B*08:01; | | | |
| | HLA-B*44:02; | | | |
| | HLA-B*44:03 | | | |
| 47 | HLA-A*01:01; | DYI*ALNEDLR | 66 | DGKDYI*ALNEDLRSWT |
| | HLA-A*03:01; | | | |
| | HLA-B*07:02; | | | |
| | HLA-B*08:01 | | | |
| 48 | HLA-A*01:01 | FDSDAASQK* | 67 | FVREDSDAASQK*MEP |
| 49 | HLA-A*24:02 | DYIAL*K | 68 | DGKDYIAL*KEDL |
| 50 | HLA-B*07:02; | FDSDAASP*R | 69 | FVREDSDAASP*REEP |
| | HLA-B*08:01 | | | |
| 51 | HLA-B*07:02; | FI*SVGYVDDTQFVR | 70 | EPRFI*SVGYVDDTQFVRFDS |
| | HLA-B*08:01 | | | |
| 52 | HLA-B*44:02; | FITVGYVDDTL*FVR | 71 | EPRFITVGYVDDTL*FVRFDS |
| | HLA-B*44:03 | | | |
| 53 | HLA-B*07:02 | GHDQYAYDGK* | 72 | LLRGHDQYAYDGK*DYI |
| 54 | HLA-B*08:01 | GHNQYAYDGK* | 73 | LLRGHNQYAYDGK*DYI |
| 55 | HLA-B*44:02; | GYDQDAYDGK* | 74 | LLRGYDQDAYDGK*DYI |
| | HLA-B*44:03 | | | |
| 56 | HLA-B*07:02; | SWTAADTAAQI*TQR | 75 | DLRSWTAADTAAQI*TQRKWE |
| | HLA-B*08:01 | | | |
| 57 | HLA-B*44:02; | TNTQ*TYR | 76 | ISKTNTQ*TYRENL |
| | HLA-B*44:03 | | | |
| 58 | HLA-B*08:01; | V*AEQDR | 77 | AARV*AEQDRAYL |
| | HLA-B*44:02 | | | |
| 59 | HLA-A*01:01; | WAAVVVP*SGEEQR | 78 | FQKWAAVVVP*SGEEQRYTC |
| | HLA-A*03:01; | | | |
| | HLA-A*24:02; | | | |
| | HLA-B*07:02; | | | |
| | HLA-B*08:01; | | | |
| | HLA-B*44:02; | | | |
| | HLA-B*44:03 | | | |
| 60 | HLA-A*24:02 | YFSTSV*SRPGR | 79 | SMRYFSTSV*SRPGRGEP |
| 61 | HLA-B*07:02 | YFYTSV*SRPGR | 80 | SMRYFYTSV*SRPGRGEP |
| 62 | HLA-B*44:02; | YYNQSEAGSHIIQ*R | 81 | ALRYYNQSEAGSHIIQ*RMYG |
| | HLA-B*44:03 | | | |

Table 5: Further peptide/protein sequences

| | | |
|---|---|---|
| 35 | HLA A*02:01, | MAVMAPRTLLLLLSGALALTQTWAGSHSMRYFFTSVSRPGRGEPRFIAVGYVDDTQFV |
| | UniProt ID | RFDSDAASQRMEPRAPWIEQEGPEYWDQETRNVKAQSQTDRVDLGTLRGYYNQSEAGS |
| | P01892 | HTIQIMYGCDVGSDGRFLRGYRQDAYDGKDYIALNEDLRSWTAADMAAQITKRKWEAA |
| | | HEAEQLRAYLDGTCVEWLRRYLENGKETLQRTDPPKTHMTHHPISDHEATLRCWALGF |
| | | YPAEITLTWQRDGEDQTQDTELVETRPAGDGTFQKWAAVVVPSGEEQRYTCHVQHEGL |
| | | PKPLTLRWELSSQPTIPIVGIIAGLVLLGAVITGAVVAAVMWRRKSSDRKGGSYTQAA |
| | | SSDSAQGSDVSLTACKV |
| 36 | beta-2- | MSRSVALAVLALLSLSGLEAIQRTPKIQVYSRHPAENGKSNFLNCYVSGFHPSDIEVD |
| | microglo- | LLKNGERIEKVEHSDLSFSKDWSFYLLYYTEFTPTEKDEYACRVNHVTLSQPKIVKWD |
| | bulin (βm), | RDM |
| | Uniprot ID | |
| | P61769 | |
| 37 | Histone H2A | MSGRGKQGGKARAKAKTRSSRAGLQFPVGRVRRLLRKGNYAERVGAGAPVYLAAVLEY |
| | UniProt ID | LTAEILELAGNAARDNKKTRIIPRHLQLAIRNDEELNKLLGKVTIAQGGVLPNIQAVL |
| | B2R5B3 | LPKKTESHHKAKGK |
| 38 | Histone H2B | MPDPAKSAPAPKKGSKKAVTKVQKKDGKKRKRSRKESYSVYVYKVLKQVHPDTGISSK |
| | UniProt ID | AMGIMNSFVNDIFERIAGEASRLAHYNKRSTITSREIQTAVRLLLPGELAKHAVSEGT |
| | B4DR52 | KAVTKYTSSNPRNLSPTKPGGSEDRQPPPSQLSAIPPFCLVLRAGIAGQV |
| 39 | Histone H4 | KPAIRRLARRGGVKRISGLIYEETRGVLKVFLENVIRDAVTYT |
| | UniProt ID | |
| | Q6B823 | |
| 40 | Seq. of | PLVEEPQNLIKQNCELFEQLGEYKFQNALLV |
| | template | |
| | protein | |
| 41 | Peptide for IS | QNCELFEQLGEYK |
| | (internal | |
| | standard) | |
| 42 | Sequence of | TLFGDKLCTVATLRETYGE |
| | template | |
| | protein | |
| 43 | Peptide for IS | LCTVATLR |
| | (internal | |
| | standard) | |

B stands for methionine sulfoxide (MetO), which may be used to replace Methionine. The underlined AA residues show the overhangs (see text). The asterisks stand behind amino acid residues which are optionally isotopically labelled.

1. A method for the absolute quantification of one or more MHC molecules in a test sample comprising at least one cell, the method comprising at least the steps of:
a) homogenizing the sample,
b) adding an internal standard comprising at least one peptide to the sample,
c) digesting the homogenized sample with a protease, before or after addition of the internal standard,
d) subjecting the digested sample to a step of chromatography and/or spectrometry analysis, and
e) quantifying the one or more MHC molecules in the test sample.

2. The method according to claim 1, wherein the protease used for digesting the sample is trypsin.

3. The method according to claim 1, further comprising the step of determining the total protein concentration in the sample prior to digestion.

4. The method according to claim 1, wherein prior to or after homogenization, the sample is not treated with, or obtained by, immunoprecipitation.

5. The method according to claim 1, wherein the test sample is selected from the group consisting of
an extract of a biological sample comprising proteins
a primary, non-cultured sample, and
sample obtained from one or more cell lines.

6. The method according to claim 5, wherein the primary sample is selected from the group consisting of a tissue sample, a blood sample, a tumor sample, and a sample of an infected tissue.

7. The method according to claim 1, wherein the MHC is MHC class I (MHC-I), optionally at least one HLA allotype selected from the group consisting of HLA-A*02:01; HLA-A*01:01; HLA-A*03:01; HLA-A*24:02; HLA-B*07:02; HLA-B*08:01; HLA-B*44:02 and HLA-B*44:03.

8.-19. (canceled)

20. The method according to claim 1, wherein the at least one peptide in the internal standard comprises an overhang of amino acids at the N-terminus and/or at the C-terminus, wherein the overhang of amino acids comprises a protease cleavage site.

21. The method according to claim 1, wherein the at least one peptide comprises the sequence corresponding to a stretch, domain or epitope of beta-2-microglobulin β2m).

22. The method according to claim 1, further comprising the step of determining the total cell count in the sample.

23. The method according to claim 1, wherein the at least one peptide comprises the sequence corresponding to a stretch, domain, or epitope of one or more proteins the abundance of which is proportional to the total number of cells in the sample, optionally wherein the at least one peptide the abundance of which is proportional to the total number of cells in the sample is a histone comprising, histone H2A, histone H2B, or histone H4.

24. The method according to claim 1, wherein the internal standard is added to the sample prior to the step of digesting the homogenized sample with a protease.

25. The method according to claim 1, the at least one peptide in the internal standard is labelled.

26. The method according to claim 25, wherein one amino acid in the at least one peptide in the internal standard is isotopically labelled by incorporation of ¹³C and/or ¹⁵N during synthesis.

27. The method according to claim 1, further comprising establishing a calibration routine, comprising the steps of
providing at least two calibration samples, the samples comprising a MHC molecule standard at varying concentrations, and, added thereto, internal standard at a fixed concentration,
digesting the calibration sample with a protease, before or after addition of the internal standard,
purifying the calibration sample obtained by the digestion,
subjecting the digested sample to a step of chromatography and/or spectrometry analysis

28. The method according to claim 27, wherein
a) the MHC molecule standard is a HLA monomer, and/or
b) the calibration samples further comprise yeast protein lysate

29. The method according to claim 27, further comprising generating a calibration curve based on the ratio of the spectrometry signals of the peptides derived from digestion of the MHC molecule standard vs. the peptides from the internal standard.

30. The method according to claim 1, wherein the concentration of the one or more MHC molecules is calculated based on the normalized protein concentration.

31. The method according to claim 1, wherein the concentration of the one or more MHC molecules vs. the test sample volume is calculated based on the total protein concentration in the test sample prior to digestion.

32. The method according to claim 1, wherein the number of the one or more MHC molecules per cell in the test sample is calculated based on the total cell count in the sample.

33.-54. (canceled)


SEQUENCE LISTING (81 sequences)

SEQ ID NO: 1 - protein, 11 aa, artificial sequence
  peptides and proteins for MHC quantification
YFFTSVSRPGR

SEQ ID NO: 2 - protein, 14 aa, artificial sequence
  peptides and proteins for MHC quantification
FIAVGYVDDTQFVR

SEQ ID NO: 3 - protein, 9 aa, artificial sequence
  peptides and proteins for MHC quantification
FDSDAASQR

SEQ ID NO: 4 - protein, 17 aa, artificial sequence
  peptides and proteins for MHC quantification
APWIEQEGPEYWDGETR

SEQ ID NO: 5 - protein, 7 aa, artificial sequence
  peptides and proteins for MHC quantification
VDLGTLR

SEQ ID NO: 6 - protein, 10 aa, artificial sequence
  peptides and proteins for MHC quantification
GYHQYAYDGK

SEQ ID NO: 7 - protein, 13 aa, artificial sequence
  peptides and proteins for MHC quantification
SWTAADBAAQTTK

SEQ ID NO: 8 - protein, 11 aa, artificial sequence
  peptides and proteins for MHC quantification
WEAAHVAEQLR

SEQ ID NO: 9 - protein, 24 aa, artificial sequence
  peptides and proteins for MHC quantification
DGEDQTQDTELVETRPAGDGTFQK

SEQ ID NO: 10 - protein, 13 aa, artificial sequence
  peptides and proteins for MHC quantification
WAAVVVPSGQEQR

SEQ ID NO: 11 - protein, 10 aa, artificial sequence
  peptides and proteins for MHC quantification
VEHSDLSFSK

SEQ ID NO: 12 - protein, 10 aa, artificial sequence
  peptides and proteins for MHC quantification
VNHVTLSQPK

SEQ ID NO: 13 - protein, 9 aa, artificial sequence
  peptides and proteins for MHC quantification
AGLQFPVGR

SEQ ID NO: 14 - protein, 9 aa, artificial sequence
  peptides and proteins for MHC quantification
LLLPGELAK

SEQ ID NO: 15 - protein, 10 aa, artificial sequence
  peptides and proteins for MHC quantification
ISGLIYEETR

SEQ ID NO: 16 - protein, 8 aa, artificial sequence
  peptides and proteins for MHC quantification
VFLENVIR

SEQ ID NO: 17 - protein, 12 aa, artificial sequence
  peptides and proteins for MHC quantification
TVTABDVVYALK

SEQ ID NO: 18 - protein, 17 aa, artificial sequence
  peptides and proteins for MHC quantification
SMRYFFTSVSRPGRGEP

SEQ ID NO: 19 - protein, 20 aa, artificial sequence
  peptides and proteins for MHC quantification
EPRFIAVGYVDDTQFVRFDS

SEQ ID NO: 20 - protein, 15 aa, artificial sequence
  peptides and proteins for MHC quantification
FVRFDSDAASQRMEP

SEQ ID NO: 21 - protein, 23 aa, artificial sequence
  peptides and proteins for MHC quantification
EPRAPWIEQEGPEYWDGETRKVK

SEQ ID NO: 22 - protein, 13 aa, artificial sequence
  peptides and proteins for MHC quantification
THRVDLGTLRGYY

SEQ ID NO: 23 - protein, 16 aa, artificial sequence
  peptides and proteins for MHC quantification
FLRGYHQYAYDGKDYI

SEQ ID NO: 24 - protein, 19 aa, artificial sequence
  peptides and proteins for MHC quantification
DLRSWTAADBAAQTTKHKW

SEQ ID NO: 25 - protein, 17 aa, artificial sequence
  peptides and proteins for MHC quantification
KHKWEAAHVAEQLRAYL

SEQ ID NO: 26 - protein, 30 aa, artificial sequence
  peptides and proteins for MHC quantification
WQRDGEDQTQDTELVETRPAGDGTFQKWAA

SEQ ID NO: 27 - protein, 19 aa, artificial sequence
  peptides and proteins for MHC quantification
FQKWAAVVVPSGQEQRYTC

SEQ ID NO: 28 - protein, 16 aa, artificial sequence
  peptides and proteins for MHC quantification
IEKVEHSDLSFSKDWS

SEQ ID NO: 29 - protein, 16 aa, artificial sequence
  peptides and proteins for MHC quantification
ACRVNHVTLSQPKIVK

SEQ ID NO: 30 - protein, 15 aa, artificial sequence
  peptides and proteins for MHC quantification
SSRAGLQFPVGRVHR

SEQ ID NO: 31 - protein, 15 aa, artificial sequence
  peptides and proteins for MHC quantification
AVRLLLPGELAKHAV

SEQ ID NO: 32 - protein, 16 aa, artificial sequence
  peptides and proteins for MHC quantification
VKRISGLIYEETRGVL

SEQ ID NO: 33 - protein, 14 aa, artificial sequence
  peptides and proteins for MHC quantification
VLKVFLENVIRDAV

SEQ ID NO: 34 - protein, 18 aa, artificial sequence
  peptides and proteins for MHC quantification
KRKTVTABDVVYALKRQG

SEQ ID NO: 35 - protein, 365 aa, artificial sequence
  peptides and proteins for MHC quantification
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 36 - protein, 119 aa, artificial sequence
  peptides and proteins for MHC quantification
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 37 - protein, 130 aa, artificial sequence
  peptides and proteins for MHC quantification
MSGRGKQGGKARAKAKTRSSRAGLQFPVGRVRRLLRKGNYAERVGAGAPVYLAAVLEYLTAEILELAGNAARDNKKTRIIPRHLQLAIRNDEELNKLLGKVTIAQGGVLPNIQAVLLPKKTESHHKAKGK

SEQ ID NO: 38 - protein, 166 aa, artificial sequence
  peptides and proteins for MHC quantification
MPDPAKSAPAPKKGSKKAVTKVQKKDGKKRKRSRKESYSVYVYKVLKQVHPDTGISSKAMGIMNSFVNDIFERIAGEASRLAHYNKRSTITSREIQTAVRLLLPGELAKHAVSEGTKAVTKYTSSNPRNLSPTKPGGSEDRQPPPSQLSAIPPFCLVLRAGIAGQV

SEQ ID NO: 39 - protein, 43 aa, artificial sequence
  peptides and proteins for MHC quantification
KPAIRRLARRGGVKRISGLIYEETRGVLKVFLENVIRDAVTYT

SEQ ID NO: 40 - protein, 31 aa, artificial sequence
  peptides and proteins for MHC quantification
PLVEEPQNLIKQNCELFEQLGEYKFQNALLV

SEQ ID NO: 41 - protein, 13 aa, artificial sequence
  peptides and proteins for MHC quantification
QNCELFEQLGEYK

SEQ ID NO: 42 - protein, 19 aa, artificial sequence
  peptides and proteins for MHC quantification
TLFGDKLCTVATLRETYGE

SEQ ID NO: 43 - protein, 8 aa, artificial sequence
  peptides and proteins for MHC quantification
LCTVATLR

SEQ ID NO: 44 - protein, 7 aa, artificial sequence
  peptides and proteins for MHC quantification
ANLGTLR

SEQ ID NO: 45 - protein, 18 aa, artificial sequence
  peptides and proteins for MHC quantification
APWIEQEGPEYWDEETGK

SEQ ID NO: 46 - protein, 14 aa, artificial sequence
  peptides and proteins for MHC quantification
APWIEQEGPEYWDR

SEQ ID NO: 47 - protein, 10 aa, artificial sequence
  peptides and proteins for MHC quantification
DYIALNEDLR

SEQ ID NO: 48 - protein, 9 aa, artificial sequence
  peptides and proteins for MHC quantification
FDSDAASQK

SEQ ID NO: 49 - protein, 6 aa, artificial sequence
  peptides and proteins for MHC quantification
DYIALK

SEQ ID NO: 50 - protein, 9 aa, artificial sequence
  peptides and proteins for MHC quantification
FDSDAASPR

SEQ ID NO: 51 - protein, 14 aa, artificial sequence
  peptides and proteins for MHC quantification
FISVGYVDDTQFVR

SEQ ID NO: 52 - protein, 14 aa, artificial sequence
  peptides and proteins for MHC quantification
FITVGYVDDTLFVR

SEQ ID NO: 53 - protein, 10 aa, artificial sequence
  peptides and proteins for MHC quantification
GHDQYAYDGK

SEQ ID NO: 54 - protein, 10 aa, artificial sequence
  peptides and proteins for MHC quantification
GHNQYAYDGK

SEQ ID NO: 55 - protein, 10 aa, artificial sequence
  peptides and proteins for MHC quantification
GYDQDAYDGK

SEQ ID NO: 56 - protein, 14 aa, artificial sequence
  peptides and proteins for MHC quantification
SWTAADTAAQITQR

SEQ ID NO: 57 - protein, 7 aa, artificial sequence
  peptides and proteins for MHC quantification
TNTQTYR

SEQ ID NO: 58 - protein, 6 aa, artificial sequence
  peptides and proteins for MHC quantification
VAEQDR

SEQ ID NO: 59 - protein, 13 aa, artificial sequence
  peptides and proteins for MHC quantification
WAAVVVPSGEEQR

SEQ ID NO: 60 - protein, 11 aa, artificial sequence
  peptides and proteins for MHC quantification
YFSTSVSRPGR

SEQ ID NO: 61 - protein, 11 aa, artificial sequence
  peptides and proteins for MHC quantification
YFYTSVSRPGR

SEQ ID NO: 62 - protein, 14 aa, artificial sequence
  peptides and proteins for MHC quantification
YYNQSEAGSHIIQR

SEQ ID NO: 63 - protein, 13 aa, artificial sequence
  peptides and proteins for MHC quantification
TDRANLGTLRGYY

SEQ ID NO: 64 - protein, 24 aa, artificial sequence
  peptides and proteins for MHC quantification
EPRAPWIEQEGPEYWDEETGKVKA

SEQ ID NO: 65 - protein, 20 aa, artificial sequence
  peptides and proteins for MHC quantification
EPRAPWIEQEGPEYWDRNTQ

SEQ ID NO: 66 - protein, 16 aa, artificial sequence
  peptides and proteins for MHC quantification
DGKDYIALNEDLRSWT

SEQ ID NO: 67 - protein, 15 aa, artificial sequence
  peptides and proteins for MHC quantification
FVRFDSDAASQKMEP

SEQ ID NO: 68 - protein, 12 aa, artificial sequence
  peptides and proteins for MHC quantification
DGKDYIALKEDL

SEQ ID NO: 69 - protein, 15 aa, artificial sequence
  peptides and proteins for MHC quantification
FVRFDSDAASPREEP

SEQ ID NO: 70 - protein, 20 aa, artificial sequence
  peptides and proteins for MHC quantification
EPRFISVGYVDDTQFVRFDS

SEQ ID NO: 71 - protein, 20 aa, artificial sequence
  peptides and proteins for MHC quantification
EPRFITVGYVDDTLFVRFDS

SEQ ID NO: 72 - protein, 16 aa, artificial sequence
  peptides and proteins for MHC quantification
LLRGHDQYAYDGKDYI

SEQ ID NO: 73 - protein, 16 aa, artificial sequence
  peptides and proteins for MHC quantification
LLRGHNQYAYDGKDYI

SEQ ID NO: 74 - protein, 16 aa, artificial sequence
  peptides and proteins for MHC quantification
LLRGYDQDAYDGKDYI

SEQ ID NO: 75 - protein, 20 aa, artificial sequence
  peptides and proteins for MHC quantification
DLRSWTAADTAAQITQRKWE

SEQ ID NO: 76 - protein, 13 aa, artificial sequence
  peptides and proteins for MHC quantification
ISKTNTQTYRENL

SEQ ID NO: 77 - protein, 12 aa, artificial sequence
  peptides and proteins for MHC quantification
AARVAEQDRAYL

SEQ ID NO: 78 - protein, 19 aa, artificial sequence
  peptides and proteins for MHC quantification
FQKWAAVVVPSGEEQRYTC

SEQ ID NO: 79 - protein, 17 aa, artificial sequence
  peptides and proteins for MHC quantification
SMRYFSTSVSRPGRGEP

SEQ ID NO: 80 - protein, 17 aa, artificial sequence
  peptides and proteins for MHC quantification
SMRYFYTSVSRPGRGEP

SEQ ID NO: 81 - protein, 20 aa, artificial sequence
  peptides and proteins for MHC quantification
ALRYYNQSEAGSHIIQRMYG